(19) United States
(12) Patent Application Publication
(10) Pub. No.: US 2022/0275332 A1
(43) Pub. Date: Sep. 1, 2022

(54) T CELL PRODUCTION FROM RAG INACTIVATED IPSCS

(71) Applicant: Adaptimmune Limited, Abingdon Oxfordshire (GB)

(72) Inventors: Laura Jane Barker, Abingdon Oxfordshire (GB); Lee Carpenter, Abingdon Oxfordshire (GB); Adam Sidaway, Abingdon Oxfordshire (GB)

(21) Appl. No.: 17/630,883

(22) PCT Filed: Aug. 20, 2020
(86) PCT No.: PCT/EP2020/073400

§ 371 Date: Jan. 27, 2022

(30) Foreign Application Priority Data
Aug. 20, 2019 (GB) 1911953.6

(51) Int. Cl. C12N 5/0783; C12N 15/86; C12N 5/0789; C12N 5/10; A61P 35/00

(52) U.S. Cl. CPC C12N 5/0638; C12N 15/86; C12N 5/0647; C12N 5/10; A61P 35/00; C12N 2501/125; C12N 2501/155; C12N 2501/16; C12N 2501/165; C12N 2506/11; C12N 2506/45

(57) ABSTRACT

This invention relates to the differentiation of recombination activating gene (RAG) inactivated progenitor cells into T cells through the expression of an exogenous T Cell Receptor (TCR). A population of T cells may be produced by a method that comprises (i) differentiating a population of RAG inactivated induced pluripotent stem cells (IPSCs) into mesoderm cells, (ii) differentiating the mesoderm cells (MCs) to produce a population of haemogenic endothelial cells (HECs), (iii) differentiating the HECs into a population of haematopoietic progenitor cells (HPCs), (iv) differentiating the population of HECs into progenitor T cells; and (v) maturing the progenitor T cells to produce a population of double positive CD4+CD8+ T cells. The method may further comprise introducing a heterologous nucleic acid encoding an antigen receptor, such as a T Cell Receptor (TCR) or chimeric antigen receptor (CAR), into one of the RAG inactivated (a) IPSCs (b) MCs (c) HECs (d) HPCs or (e) the progenitor T cells. This may be useful for example in the production of T cells for immunotherapy.

### FIELD

This invention relates to methods of producing T cells from RAG inactivated progenitor cells, such as induced pluripotent stem cells (iPSCs).

### BACKGROUND

Immunotherapeutics are poised to transform the cancer treatment landscape with the promise of long-term survival (McDermott et al., Cancer Treat Rev. 2014 October; 40(9): 1056-64). There is a clear unmet medical need for new immunomodulatory drugs to expand patient population and range of tumor types. In addition, new agents are needed to enhance the magnitude and duration of anti-tumor responses. The development of these agents has been possible because of the in-depth understanding of the basic principles controlling T-cell immunity over the last two decades (Sharma and Allison, Cell. 2015 Apr. 9; 161(2): 205-14). This typically requires tumor specific CD4+ and CD8+ T-cells recognising tumor-associated peptide antigens presented by MHC molecules. Different vaccination strategies and adoptive transfer of ex vivo expanded tumor infiltrated lymphocytes have in some cases demonstrated the ability of tumor specific T-cells to treat late stage cancer (Rosenberg et al., Nat Med. 2004 September; 10(9): 909-15).

However, current adoptive T cell therapies are limited by a lack of suitable patient and tumor-specific T cells and there is a need for therapeutically sufficient and functional antigen-specific T cells for effective use in immunotherapy.

The expression of endogenous T cell receptors (TCRs) may cause T cells to exert off-target toxicity when administered therapeutically. Whilst the expression of endogenous TCRs may in principle be prevented by the inactivation of the recombination activating gene 1 (RAG1) and/or recombination activating gene 2 (RAG2), this inactivation also blocks the differentiation of precursor cells into lymphopoietic lineage (Mombaerts et al (1992) Cell 68 (5) 869-877).

### SUMMARY

The present inventors have recognised that the capacity of RAG inactivated progenitor cells to differentiate into T cells can be restored by the expression of an exogenous T Cell Receptor (TCR). This may be useful for example in the production of T cells for immunotherapy.

A first aspect of the invention provides a method of producing a population of T cells which may comprise;
(i) differentiating a population of induced pluripotent stem cells (iPSCs) into mesoderm cells (MCs),
(ii) differentiating the MCs to produce a population of haemogenic endothelial cells (HECs),
(iii) differentiating the HECs into a population of haematopoietic progenitor cells (HPCs),
(iv) differentiating the population of HPCs into progenitor T cells; and
(v) maturing the progenitor T cells to produce a population of double positive CD4+CD8+ T cells,
wherein the method comprises
(a) inactivating RAG in one of the (a) iPSCs, (b) MCs and (c) HECs; and
(b) introducing a heterologous nucleic acid encoding an antigen receptor, such as a T Cell Receptor (TCR) or chimeric antigen receptor (CAR), into one of the RAG inactivated (a) iPSCs, (b) MCs (c) HECs (d) HPCs or (e) the progenitor T cells.

For example, a method of the first aspect may comprise;
(i) differentiating a population of RAG inactivated (RAG−/−) induced pluripotent stem cells (iPSCs) into mesoderm cells
(ii) differentiating the mesoderm cells (MCs) to produce a population of haemogenic endothelial cells (HECs),
(iii) differentiating the HECs into a population of haematopoietic progenitor cells (HPCs),
(iv) differentiating the population of HPCs into progenitor T cells; and
(v) maturing the progenitor T cells to produce a population of double positive CD4+CD8+ T cells,
wherein the method comprises introducing a heterologous nucleic acid encoding an antigen receptor, such as a T Cell Receptor (TCR) or chimeric antigen receptor (CAR), into one of the RAG inactivated cells selected from, (a) iPSCs, (b) MCs (c) HECs (d) HPCs and (e) the progenitor T cells.

A method of the first aspect may further comprise;
(vi) activating and expanding the double positive CD4+CD8+ T cells to produce a population of CD8+ T cells or a population of CD4+ T cells.

A second aspect of the invention provides a population of T cells produced by a method of the first aspect. The T cells may be RAG inactivated (RAG−/−) and may display expression of a heterologous antigen receptor and no expression of an endogenous TCR.

A third aspect of the invention provides a pharmaceutical composition comprising a population of T cells of the second aspect and a pharmaceutically acceptable excipient.

A fourth aspect of the invention provides a method of treatment comprising administering a therapeutically effective dose of a population of T cells of the second aspect to an individual in need thereof.

These and other aspects and embodiments of the invention are described in more detail below.

### BRIEF DESCRIPTION OF FIGURES

FIG. 1 shows a schematic view of an example of a six-stage method for generating T cells from iPSCs.

FIG. 2 shows virus titration of ADP-A2M4 Lentivirus on peripheral blood lymphocytes (PBL) and Stage 5 iPSC-derived T cells. Flow cytometry plots show % Vα24+CD3+ T cells with increasing dilutions of ADP-A2M4 lentivirus in a) PBL and b) iPSC-derived T cells (HCP547). Populations are gated on total live cells. c) Surface expression of CD3 correlates with ADP-A2M4 transduction efficiency in iPSC-derived T cells. Populations gated on total live cells. d) Expression of Vα24 on CD3+ T cell populations in lentivirally transduced iPSC-derived T cells. Populations gated on CD3+ T cells. Data representative of duplicates.

FIG. 3 shows percentages of CD3+ cells in transduced and non-transduced populations of CGT3 RAG−/− and CGT3 wild type iPSC lines with or without transduction of ADP-A2M4 TCR. Transduction of CD7+/CD5+ progenitors is shown to result in an increased proportion of CD3 expressing cells.

FIG. 4 shows total cell numbers of CGT3 RAG−/− with or without transduction of ADP-A2M4 TCR. T-cell progenitor cells at the CD7+/CD5+ stage were transduced with ADP-A2M4 TCR and cell counts performed at regular intervals.

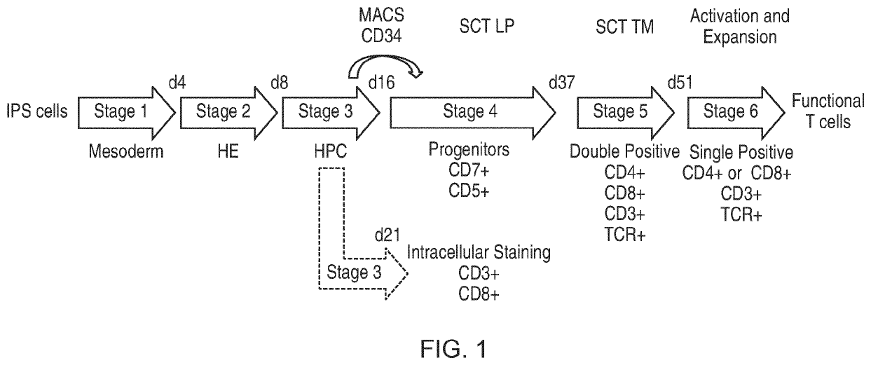

### DETAILED DESCRIPTION

This invention relates to the finding that transduction of RAG inactivated cells with nucleic acid encoding an exogenous T cell receptor (TCR) overcomes the differentiation arrest caused by RAG inactivation and allows the production of T cells without endogenous TCRs. This may be useful, for example, in the generation of T cells for immunotherapy.

iPSCs are pluripotent cells which are derived from non-pluripotent, fully differentiated donor or antecedent cells. iPSCs are capable of self-renewal in vitro and exhibit an undifferentiated phenotype and are potentially capable of differentiating into any foetal or adult cell type of any of the three germ layers (endoderm, mesoderm and eectoderm). The population of iPSCs may be clonal i.e. genetically identical cells descended from a single common ancestor cell.

iPSCs may express one or more of the following pluripotency associated markers: POU5f1 (Oct4), Sox2, Alkaline Phosphatase, SSEA-3, Nanog, SSEA-4, Tra-1-60, KLF4 and c-myc, preferably one or more of POU5f1, NANOG and SOX2. An iPSC may lack markers associated with specific differentiative fates, such as Bra, Sox17, FoxA2, αFP, Sox1, NCAM, GATA6, GATA4, Hand1 and CDX2. In particular, an iPSC may lack markers associated with endodermal fates.

Preferably, the iPSCs are human IPSCs (hiPSCs).

In some embodiments, iPSCs may be gene edited, for example to inactivate or delete HLA genes or other genes associated with immunogenicity or GVHD, or optionally to include nucleic acids encoding exogenous antigen receptor, for example exogenous TCR, CAR or NKCR.

iPSCs may be derived or reprogramed from donor cells, which may be somatic cells or other antecedent cells obtained from a source, such as a donor individual. The donor cells may be mammalian, preferably human cells. Suitable donor cells include adult fibroblasts and blood cells, for example peripheral blood cells, such as HPCs or mononuclear cells.

Suitable donor cells for reprogramming into iPSCs as described herein may be obtained from a donor individual. In some embodiments, the donor individual may be the same person as the recipient individual to whom the T cells will be administered following production as described herein (autologous treatment). In other embodiments, the donor individual may be a different person to the recipient individual to whom the T cells will be administered following production as described herein (allogeneic treatment). For example, the donor individual may be a healthy individual who is human leukocyte antigen (HLA) matched (either before or after donation) with a recipient individual suffering from cancer. In other embodiments, the donor individual may not be HLA matched with the recipient individual. Preferably, the donor individual may be a neonate (new-born), for example the donor cells may be obtained from a sample of umbilical cord blood.

Suitable donor individuals are preferably free of communicable viral (e.g. HIV, HPV, CMV) and adventitious agents (e.g. bacteria, mycoplasma), and free of known genetic abnormalities.

In some embodiments, a population of peripheral blood cells, such as HPCs, for reprogramming may be isolated from a blood sample, preferably an umbilical cord sample, obtained from the donor individual. Suitable methods for the isolation of HPCs and other peripheral blood cells, are well-known in the art and include, for example magnetic activated cell sorting (see, for example, Gaudernack et al 1986 J Immunol Methods 90 179), fluorescent activated cell sorting (FACS: see for example, Rheinherz et al (1979) PNAS 76 4061), and cell panning (see for example, Lum et al (1982) Cell Immunol 72 122). HPCs may be identified in a sample of blood cells by expression of CD34. In other embodiments, a population of fibroblasts for reprogramming may be isolated from a skin biopsy following dispersal using collagenase or trypsin and out-growth in appropriate cell culture conditions.

In some embodiments, IPSCs may be derived from antigen-specific T cells. For example, the T cells may comprise nucleic acid encoding αβ TCRs that bind to an antigen, such as a tumor antigen, displayed in complex with a class I MHC. Antigen-specific T cells for use in the generation of iPSCs may be obtained by screening a diverse population of T cells with peptide epitopes from the target antigen displayed on a class I or II MHC molecule on the surface of an antigen presenting cell, such as a dendritic cell, or by isolating from a tumour sample from a cancer patient.

Donor cells are typically reprogrammed into iPSCs by the introduction of reprogramming factors, such as Oct4, Sox2 and Klf4 into the cell. The reprogramming factors may be proteins or encoding nucleic acids and may be introduced into the differentiated cells by any suitable technique, including plasmid, transposon or more preferably, viral transfection or direct protein delivery. Other reprogramming factors, for example Klf genes, such as Klf-1, -2, -4 and -5; Myc genes such as C-myc, L-myc and N-myc; Nanog; SV40 Large T antigen; Lin28; and short hairpins (shRNA) targeting genes such as p53, may also be introduced into the cell to increase induction efficiency. Following introduction of the reprogramming factors, the donor cells may be cultured. Cells expressing pluripotency markers may be isolated and/or purified to produce a population of iPSCs. Techniques for the production of iPSCs are well-known in the art (Yamanaka et al Nature 2007; 448:313-7; Yamanaka 6 2007 Jun. 7; 1(1):39-49; Kim et al Nature. 2008 Jul. 31; 454(7204):646-50; Takahashi Cell. 2007 Nov. 30; 131(5):861-72. Park et al Nature. 2008 Jan. 10; 451(7175):141-6; Kimet et al Cell Stem Cell. 2009 Jun. 5; 4(6):472-6; Vallier, L., et al. Stem Cells, 2009. 9999(999A): p. N/A; Baghbaderani et al 2016; Stem Cell Rev. 2016 August; 12(4):394-420; Baghbaderani et al. (2015) Stem Cell Reports, 5(4), 647-659).

Conventional techniques may be employed for the culture and maintenance of iPSCs (Vallier, L. et al Dev. Biol. 275, 403-421 (2004), Cowan, C. A. et al. N. Engl. J. Med. 350, 1353-1356 (2004), Joannides, A. et al. Stem Cells 24, 230-235 (2006) Klimanskaya, I. et al. Lancet 365, 1636-1641 (2005), Ludwig, T. E. et al. Nat. Biotechnol. 24, 185-187 (2006)). iPSCs for use in the present methods may be grown in defined conditions or on feeder cells. For example, iPSCs may be conventionally cultured in a culture dish on a layer of feeder cells, such as irradiated mouse embryonic fibroblasts (MEF), at an appropriate density (e.g. 10⁵ to 10⁶ cells/60 mm dish), or on an appropriate substrate, in a feeder conditioned or defined iPSC maintenance medium. iPSCs for use in the present methods may be passaged by enzymatic or mechanical means. In some embodiments, iPSCs may be passaged on matrigel™ or an ECM protein, such as vitronectin, in an iPSC maintenance medium, such as mTeSR1 (StemCell Technologies) or E8 flex (Life Thermo) culture medium.

Recombination activating gene 1 (RAG1) and recombination activating gene 2 (RAG2) mediate the V(D)J recombination of immunoglobulin and T cell receptor genes. B and T cell differentiation is arrested at an early stage in mice deficient in RAG1 and/or RAG2 and RAG deficient mice lack mature B and T lymphocytes (Mombaerts et al (1992) Cell 68 (5) 869-877; Shinkai Yet al (1992) Cell. 68 (5): 855-67).

RAG1 (Gene ID 5896; also called RAG-1) is preferably human RAG1 and may have the amino acid sequence of database entry NP_00439.1 and may be encoded by the nucleic acid sequence of database entry NM_000448.2. RAG2 (Gene ID 5897; also called RAG-2) is preferably human RAG2 and may have the amino acid sequence of database entry NP_000527.2, NM_001243785.2 or NM_001243786.1, and may be encoded by the nucleic acid sequence of database entry NM_000536.4, NP_001230714.1, or NP_001230715.1.

RAG inactivated cells (which may be termed RAG−/− or null) are cells which have been engineered to inactivate RAG1 and/or RAG2 i.e. the RAG1 and/or RAG2 coding sequence or regulatory region has been deleted or otherwise mutated to prevent the expression of active protein. RAG inactivated iPSCs are unable to express active forms of one or both of RAG1 and RAG2 and are cannot therefore support the V(D)J recombination of immunoglobulin and T cell receptor genes. Because they lack the ability to generate active immunoglobulin and T cell receptor genes through recombination, RAG inactivated cells cannot differentiate into lymphopoietic lineages.

A method may comprise inactivating one or both of RAG1 and RAG2 to produce RAG inactivated cells. For example, the genes or their regulatory regions may be deleted by recombinant techniques. Suitable methods for the inactivation of genes are well-known in the art and may include AAV mediated gene editing (Hirsch et al 2014 Methods Mol Biol 1114 291-307) and CRISPR/Cas9 gene editing. In other embodiments, expression of RAG genes may be suppressed using a suppressor nucleic acid, such as siRNA or shRNA or RAG activity may be inhibited using an inhibitor. RAG1 and RAG2 may be inactivated by any convenient method.

In some embodiments, iPSCs for use in the methods described herein may be RAG inactivated iPSCs (RAG−/− iPSCs).

In other embodiments, RAG proficient (RAG+/+) iPSCs may be employed in the methods described herein and RAG may be inactivated at the MC or HEC stage. For example, a method may comprise (a) inactivating RAG in the mesoderm cells to produce population of RAG inactivated mesoderm cells or (b) inactivating RAG in the HECs to produce population of RAG inactivated HECs. The RAG inactivated cells may be differentiated to produce T cells as described herein. The heterologous nucleic acid encoding the antigen receptor may be introduced following said RAG inactivation.

T cells may be produced from a population of iPSCs by a method comprising;
(i) differentiating the population of iPSCs into mesoderm cells (MCs),
(ii) differentiating the MCs to produce a population of haemogenic endothelial cells (HECs),
(iii) differentiating the HECs into a population of haematopoietic progenitor cells (HPCs),
(iv) differentiating the population of HPCs into progenitor T cells; and
(v) maturing the progenitor T cells to produce a population of double positive CD4+CD8+ T cells.

The cells may be RAG inactivated before introduction of the heterologous nucleic acid. For example, the iPSCs may be RAG inactivated or RAG may be inactivated in the MCs or HECs.

The heterologous nucleic acid encoding the antigen receptor may be introduced into the RAG inactivated cells before step (i), or after any one of steps (i) to (iv). For example, one of the (a) iPSCs, (b) MCs (c) HECs (d) HPCs or (e) progenitor T cells may be transfected or transduced with a heterologous nucleic acid encoding an antigen receptor, such as a T Cell Receptor (TCR) or chimeric antigen receptor.

T cells produced as described herein may express an antigen receptor that binds a target antigen. For example, the antigen receptor may bind specifically to cancer cells that express a tumor antigen. The T cells may be useful for example in immunotherapy, as described below. T cells are produced in the methods described herein from RAG inactivated precursor cells. Because the RAG inactivated cells do not support V(D)J recombination, the T cells produced herein do not contain functional endogenous TCR genes and do not express endogenous TCRs.

The T cells express an exogenous antigen receptor that is encoded by heterologous nucleic acid introduced into the cells during production by a recombinant technique, such as gene editing or transduction. The antigen receptor is not naturally expressed by the cells (i.e. the antigen receptor is exogenous or heterologous). Heterologous antigen receptors may include T cell receptors (TCRs), such as αβTCR heterodimers, γδ TCR heterodimers and NKT receptors; or chimeric antigen receptors (CARs).

The antigen receptor may be a T cell receptor (TCR). TCRs are disulphide-linked membrane anchored heterodimeric proteins that comprise highly variable alpha (α) and beta (β) chains expressed as a complex with invariant CD3 chain molecules. T cells expressing this type of TCRs (αβ TCRs) may be referred to as αβ (or αβ) T cells. A minority of T cells express an alternative TCR comprising variable gamma (γ) and delta (δ) chains and are referred to as γδ T cells.

TCRs bind specifically to major histocompatibility complexes (MHC) on the surface of cells that display a peptide fragment of a target antigen. For example, TCRs may bind specifically to a major histocompatibility complex (MHC) on the surface of cancer cells that displays a peptide fragment of a tumour antigen. An MHC is a set of cell-surface proteins which allow the acquired immune system to recognise ‘foreign’ molecules. Proteins are intracellularly degraded and presented on the surface of cells by the MHC. MHCs displaying ‘foreign’ peptides, such a viral or cancer associated peptides, are recognised by T cells with the appropriate TCRs, prompting cell destruction pathways. MHCs on the surface of cancer cells may display peptide fragments of tumour antigen i.e. an antigen which is present on a cancer cell but not the corresponding non-cancerous cell. T cells which recognise these peptide fragments may exert a cytotoxic effect on the cancer cell.

Suitable heterologous TCR may bind specifically to class I or II MHC molecules displaying peptide fragments of a target antigen. For example, the T cells may be modified to express a heterologous TCR that binds specifically to class I or II MHC molecules displaying peptide fragments of a tumour antigen expressed by the cancer cells in a cancer patient, or the T cells may be modified to express a heterologous TCR that specifically binds to a tumour antigen or peptide fragment thereof expressed by the cancer cells in the absence of MHC presentation in a cancer patient. Tumour antigens expressed by cancer cells in the cancer patient may identified using standard techniques. Preferred tumour antigens include NY-ESO1, PRAME, alpha-fetoprotein (AFP), MAGE A4, MAGE A1, MAGE A10 and MAGE B2, most preferably NY-ESO-1, MAGE-A4 and MAGE-A10.

Suitable TCRs may include unconventional TCRs, for example non-MHC dependent TCRs that bind recognize non-peptide antigens displayed by monomorphic antigen-presenting molecules, such as CD1 and MR1; NKT cell TCRs and intraepithelial lymphocyte (IEL) TCRs. In some embodiments, the TCR may recognise target antigen or peptide fragment of target antigen on the cancer cell independently of MHC presentation.

A heterologous TCR may be a synthetic or artificial TCR i.e. a TCR that does not exist in nature. For example, a heterologous TCR may be engineered to increase its affinity or avidity for a tumour antigen (i.e. an affinity enhanced TCR). The affinity enhanced TCR may comprise one or more mutations relative to a naturally occurring TCR, for example, one or more mutations in the hypervariable complementarity determining regions (CDRs) of the variable regions of the TCR α and β chains or γ and δ chains. These mutations increase the affinity of the TCR for MHCs that display a peptide fragment of a tumour antigen expressed by cancer cells. Suitable methods of generating affinity enhanced TCRs include screening libraries of TCR mutants using phage or yeast display and are well known in the art (see for example Robbins et al J Immunol (2008) 180(9):6116; San Miguel et al (2015) Cancer Cell 28 (3) 281-283; Schmitt et al (2013) Blood 122 348-256; Jiang et al (2015) Cancer Discovery 5 901). Preferred affinity enhanced TCRs may bind to cancer cells expressing one or more of the tumour antigens NY-ESO1, PRAME, alpha-fetoprotein (AFP), MAGE A4, MAGE A1, MAGE A10 and MAGE B2.

Alternatively, the antigen receptor may be a chimeric antigen receptor (CAR). CARs are artificial receptors that are engineered to contain an immunoglobulin antigen binding domain, such as a single-chain variable fragment (scFv). A CAR may, for example, comprise an scFv fused to a TCR CD3 transmembrane region and endodomain. An scFv is a fusion protein of the variable regions of the heavy (V_{H}) and light (V_{L}) chains of immunoglobulins, which may be connected with a short linker peptide of approximately 10 to 25 amino acids (Huston J. S. et al. Proc Natl Acad Sci USA 1988; 85(16):5879-5883). The linker may be glycine-rich for flexibility, and serine or threonine rich for solubility, and may connect the N-terminus of the V_{H} to the C-terminus of the V_{L}, or vice versa. The scFv may be preceded by a signal peptide to direct the protein to the endoplasmic reticulum, and subsequently the T cell surface. In the CAR, the scFv may be fused to a TCR transmembrane and endodomain. A flexible spacer may be included between the scFv and the TCR transmembrane domain to allow for variable orientation and antigen binding. The endodomain is the functional signal-transmitting domain of the receptor. An endodomain of a CAR may comprise, for example, intracellular signalling domains from the CD3 ζ-chain or from receptors such as CD28, 41 BB, or ICOS. A CAR may comprise multiple signalling domains, for example, but not limited to, CD3z-CD28-41BB or CD3z-CD28-OX40.

The CAR may bind specifically to a tumour-specific antigen expressed by cancer cells. For example, the T cells may be modified to express a CAR that binds specifically to a tumour antigen that is expressed by the cancer cells in a specific cancer patient. Tumour antigens expressed by cancer cells in the cancer patient may identified using standard techniques.

Alternatively, the antigen receptor may be an NK cell receptor (NKCR).

Expression of a heterologous antigen receptor, such as a heterologous TCR, NKCR or CAR may alter the immunogenic specificity of the T cells produced as described herein so that they recognise or display improved recognition for one or more target antigens, e.g. tumour antigens that are present on the surface of the cancer cells of an individual with cancer. In some embodiments, the T cells produced as described herein lack endogenous TCRs and may display reduced binding or no binding to cancer cells in the absence of the heterologous antigen receptor. For example, expression of the heterologous TCR may increase the affinity and/or specificity of the cancer cell binding of a T cell relative to T cells that do not express the antigen receptor.

The term “heterologous” refers to a polypeptide or nucleic acid that is foreign to a particular biological system, such as a host cell, and is not naturally present in that system. A heterologous polypeptide or nucleic acid may be introduced to a biological system by artificial means, for example using recombinant techniques. For example, heterologous nucleic acid encoding a polypeptide may be inserted into a suitable expression construct which is in turn used to transform a host cell to produce the polypeptide. A heterologous polypeptide or nucleic acid may be synthetic or artificial or may exist in a different biological system, such as a different species or cell type. An endogenous polypeptide or nucleic acid is native to a particular biological system, such as a host cell, and is naturally present in that system. A recombinant polypeptide is expressed from heterologous nucleic acid that has been introduced into a cell by artificial means, for example using recombinant techniques. A recombinant polypeptide may be identical to a polypeptide that is naturally present in the cell or may be different from the polypeptides that are naturally present in that cell.

Heterologous nucleic acid encoding a TCR may encode all the sub-units of the receptor. For example, nucleic acid encoding a TCR may comprise a nucleotide sequence encoding a TCR α chain and a nucleotide sequence encoding a TCR β chain or a nucleotide sequence encoding a TCR δ chain and a nucleotide sequence encoding a TCR γ chain. Suitable nucleotide sequences are well known in the art.

Cells may be modified to express the heterologous antigen receptor, such as a TCR or CAR, by the introduction of heterologous encoding nucleic acid into the RAG inactivated cells at any stage in the method described herein. For example, heterologous encoding nucleic acid may be introduced into the iPSCs, mesoderm, haemogenic endothelium (HE; i.e. HECs), HPCs or progenitor T cells. In some preferred embodiments, cells may be transduced with heterologous nucleic acid encoding the antigen receptor after 2 weeks culture in lymphoid expansion medium (stage 4) as described herein (i.e. when the cells are progenitor T cells). Heterologous nucleic acid encoding an antigen receptor may encode all the sub-units of the receptor. For example, nucleic acid encoding a TCR may comprise a nucleotide sequence encoding a TCR α chain and a nucleotide sequence encoding a TCR β chain, or a nucleotide sequence encoding a TCR δ chain and a nucleotide sequence encoding a TCR γ chain.

Nucleic acid may be introduced into the cells by any convenient technique. Suitable techniques for transporting the expression vector into the iPSCs, mesoderm, HE (HECs), HPCs or progenitor T cells are well known in the art and include calcium phosphate transfection, DEAE-Dextran, electroporation, liposome-mediated transfection and transduction using retrovirus or other virus, e.g. vaccinia or lentivirus Or by gene editing into a specific location. When introducing or incorporating a heterologous nucleic acid into an iPSC, HPC or progenitor T cell, certain considerations must be taken into account, well-known to those skilled in the art. The nucleic acid to be inserted should be assembled within a construct or vector which contains effective regulatory elements which will drive transcription in the T cell. Many known techniques and protocols for manipulation and transformation of nucleic acid, for example in preparation of nucleic acid constructs, introduction of DNA into cells and gene expression are described in detail in Protocols in Molecular Biology, Second Edition, Ausubel et al. eds. John Wiley & Sons, 1992. In some embodiments, nucleic acid may be introduced into the cells by gene editing. For example, a DNA double strand break (DSB) at a target site may be induced by a CRISPR/Cas9 system and the repair of the DSB may introduce the heterologous nucleic acid into the cell genome at the target site or the nucleic acid may be introducing using an rAAV vector (AAV mediated gene editing; Hirsch et al 2014 Methods Mol Biol 1114 291-307).

Suitable techniques for introducing the expression vector into the iPSCs, mesoderm, HE (HECs), HPCs or progenitor T cells are well known in the art and include calcium phosphate transfection, DEAE-Dextran, electroporation, liposome-mediated transfection, gene editing, for example AAV mediated gene editing, and transduction using retrovirus or other virus, e.g. vaccinia or lentivirus. Preferably, nucleic acid encoding the heterologous TCR may be contained in a viral vector, most preferably a gamma retroviral vector or a lentiviral vector, such as a VSVg-pseudotyped lentiviral vector. A method described herein may comprise transducing a population of cells, for example iPSCs, mesoderm, HE (HECs), HPCs or progenitor T cells, with a viral vector to produce a transduced population of genetically modified cells. The cells may be transduced by contact with a viral particle comprising the nucleic acid. Viral particles for transduction may be produced according to known methods. For example, HEK293T cells may be transfected with plasmids encoding viral packaging and envelope elements as well as a lentiviral vector comprising the coding nucleic acid. A VSVg-pseudotyped viral vector may be produced in combination with the viral envelope glycoprotein G of the Vesicular stomatitis virus (VSVg) to produce a pseudotyped virus particle. For example, solid-phase transduction may be performed without selection by culture on retronectin-coated, retroviral vector-preloaded tissue culture plates.

Differentiation and maturation of the cell populations in the steps of the methods described herein is induced by culturing the cells in a culture medium supplemented with a set of differentiation factors. The set of differentiation factors that is listed for each culture medium is preferably exhaustive and medium may be devoid of other differentiation factors. In preferred embodiments, the culture media are chemically defined media. For example, a culture medium may consist of a chemically defined nutrient medium that is supplemented with an effective amount of one or more differentiation factors, as described below. A chemically defined nutrient medium may comprise a basal medium that is supplemented with one or more serum-free culture medium supplements.

Differentiation factors are factors which modulate, for example promote or inhibit, a signalling pathway which mediates differentiation in a mammalian cell. Differentiation factors may include growth factors, cytokines and small molecules which modulate one or more of the Activin/Nodal, FGF, Wnt or BMP signalling pathways. Examples of differentiation factors include Activin/Nodal, FGFs, BMPs, retinoic acid, vascular endothelial growth factor (VEGF), stem cell factor (SCF), TGFβ ligands, GDFs, LIF, Interleukins, GSK-3 inhibitors and phosphatidylinositol 3-kinase (PI3K) inhibitors.

Differentiation factors which are used in one or more of the media described herein include TGFβ ligands, such as activin, fibroblast growth factor (FGF), bone morphogenetic protein (BMP), stem cell factor (SCF), vascular endothelial growth factor (VEGF), GSK-3 inhibitors (such as CHIR-99021), interleukins, and hormones, such as IGF-1 and angiotensin II. A differentiation factor may be present in a medium described herein in an amount that is effective to modulate a signalling pathway in cells cultured in the medium.

In some embodiments, a differentiation factor listed above or below may be replaced in a culture medium by a factor that has the same effect (i.e. stimulation or inhibition) on the same signalling pathway. Suitable factors are known in the art and include proteins, nucleic acids, antibodies and small molecules.

The extent of differentiation of the cell population during each step may be determined by monitoring and/or detecting the expression of one or more cell markers in the population of differentiating cells. For example, an increase in the expression of markers characteristic of the more differentiated cell type or a decrease in the expression of markers characteristic of the less differentiated cell type may be determined. The expression of cell markers may be determined by any suitable technique, including immunocytochemistry, immunofluorescence, RT-PCR, immunoblotting, fluorescence activated cell sorting (FACS), and enzymatic analysis. In preferred embodiments, a cell may be said to express a marker if the marker is detectable on the cell surface. For example, a cell which is stated herein not to express a marker may display active transcription and intracellular expression of the marker gene but detectable levels of the marker may not be present on the surface of the cell.

A population of partially differentiated cells that is produced by a step in the methods described herein may be cultured, maintained or expanded before the next differentiation step. Partially differentiated cells may be expanded by any convenient technique.

After each step, the population of partially differentiated cells which is produced by that step may contain 1% or more, 5% or more, 10% or more or 15% or more partially differentiated cells, following culture in the medium. If required, the population of partially differentiated cells may be purified by any convenient technique, such as MACs or FACS.

Cells may be cultured in a monolayer, in the absence of feeder cells, on a surface or substrate coated with extracellular matrix protein, such as fibronectin, laminin or collagen. Suitable techniques for cell culture are well-known in the art (see, for example, Basic Cell Culture Protocols, C. Helgason, Humana Press Inc. U.S. (15 Oct. 2004) ISBN: 1588295451; Human Cell Culture Protocols (Methods in Molecular Medicine S.) Humana Press Inc., U.S. (9 Dec. 2004) ISBN: 1588292223; Culture of Animal Cells: A Manual of Basic Technique, R. Freshney, John Wiley & Sons Inc (2 Aug. 2005) ISBN: 0471453293, Ho W Y et al J Immunol Methods. (2006) 310:40-52, Handbook of Stem Cells (ed. R. Lanza) ISBN: 0124366430) Basic Cell Culture Protocols' by J. Pollard and J. M. Walker (1997), ‘Mammalian Cell Culture: Essential Techniques’ by A. Doyle and J. B. Griffiths (1997), ‘Human Embryonic Stem Cells’ by A. Chiu and M. Rao (2003), Stem Cells: From Bench to Bedside' by A. Bongso (2005), Peterson & Loring (2012)Human Stem Cell Manual: A Laboratory Guide Academic Press and ‘Human Embryonic Stem Cell Protocols’ by K. Turksen (2006). Media and ingredients thereof may be obtained from commercial sources (e.g. Gibco, Roche, Sigma, Europa bioproducts, R&D Systems). Standard mammalian cell culture conditions may be employed for the above culture steps, for example 37° C., 5% or 21% Oxygen, 5% Carbon Dioxide. Media is preferably changed every two days and cells allowed to settle by gravity.

Cells may be cultured in a culture vessel. Suitable cell culture vessels are well-known in the art and include culture plates, dishes, flasks, bioreactors, and multi-well plates, for example 6-well, 12-well or 96-well plates.

The culture vessels are preferably treated for tissue culture, for example by coating one or more surfaces of the vessel with an extracellular matrix protein, such as fibronectin, laminin or collagen. Culture vessels may be treated for tissue culture using standard techniques, for example by incubating with a coating solution, as described herein, or may be obtained pre-treated from commercial suppliers.

In a first stage, iPSCs may be differentiated into mesoderm cells by culturing the population of iPSCs under suitable conditions to promote mesodermal differentiation. For example, the iPSCs cells may be cultured sequentially in first, second and third mesoderm induction media to induce differentiation into mesoderm cells.

A suitable first mesoderm induction medium may stimulate SMAD2 and SMAD3 mediated signalling pathways. For example, the first mesoderm induction medium may comprise activin.

A suitable second mesoderm induction medium may (i) stimulate SMAD1, SMAD2, SMAD3, SMAD5 and SMAD9 and/or SMAD1, SMAD2, SMAD3, SMAD5 and SMAD9 mediated signalling pathways and (ii) have fibroblast growth factor (FGF) activity. For example, the second mesoderm induction medium may comprise activin, preferably activin A, BMP, preferably BMP4 and FGF, preferably bFGF.

A suitable third mesoderm induction medium may (i) stimulate SMAD1, SMAD2, SMAD3, SMAD5 and SMAD9 and/or SMAD1, SMAD2, SMAD3, SMAD5 and SMAD9 mediated signalling pathways (ii) have fibroblast growth factor (FGF) activity and (iii) inhibit glycogen synthase kinase 3β. For example, the third mesoderm induction medium may comprise activin, preferably activin A, BMP, preferably BMP4, FGF, preferably bFGF, and a GSK3 inhibitor, preferably CHIR99021.

The first, second and third mesoderm induction media may be devoid of differentiation factors other than the differentiation factors set out above.

SMAD2 and SMAD3 mediated intracellular signalling pathways may be stimulated by the first, second and third mesoderm induction media through the presence in the media of a first TGFβ ligand. The first TGFβ ligand may be Activin. Activin (Activin A: NCBI Gene ID: 3624 nucleic acid reference sequence NM_002192.2 GI: 62953137, amino acid reference sequence NP_002183.1 GI: 4504699) is a dimeric polypeptide which exerts a range of cellular effects via stimulation of the Activin/Nodal pathway (Vallier et al., Cell Science 118:4495-4509 (2005)). Activin is readily available from commercial sources (e.g. Stemgent Inc. MA USA; Miltenyi Biotec Gmbh, DE). Conveniently, the concentration of Activin in a medium described herein may be from 1 to 100 Ong/ml, for example any of about 2, 3, 4, 5, 6, 7, 8, 9, 10, 12, 15, 17, 20, 25, 30, 35, 40, 45 or 50, 55, 60, 65, 70, 75, 80, 85, 90 or 95 ng/ml, preferably about 5 to 50 ng/ml.

The fibroblast growth factor (FGF) activity of the second and third mesoderm induction media may be provided by the presence of fibroblast growth factor (FGF) in the media. Fibroblast growth factor (FGF) is a protein factor which stimulates cellular growth, proliferation and cellular differentiation by binding to a fibroblast growth factor receptor (FGFR). Suitable fibroblast growth factors include any member of the FGF family, for example any one of FGF1 to FGF14 and FGF15 to FGF23. Preferably, the FGF is FGF2 (also known as bFGF, NCBI GeneID: 2247, nucleic acid sequence NM_002006.3 GI: 41352694, amino acid sequence NP_001997.4 GI: 41352695); FGF7 (also known as keratinocyte growth factor (or KGF), NCBI GeneID: 2247, nucleic acid sequence NM_002006.3 GI: 41352694, amino acid sequence NP_001997.4 GI: 41352695); or FGF10 (NCBI GeneID: 2247, nucleic acid sequence NM_002006.3 GI: 41352694, amino acid sequence NP_001997.4 GI: 41352695). Most preferably, the fibroblast growth factor is FGF2.

Conveniently, the concentration of FGF, such as FGF2 in a medium described herein may be from 0.5 to 50 ng/ml, for example any of about 0.5, 1, 2, 3, 4, 5, 6, 7, 8, 9, 10, 11, 12, 13, 14, 15, 20, 25, 30, 35, 40, 45, 50 ng/ml, preferably about 5 ng/ml. Fibroblast growth factors, such as FGF2, FGF7 and FGF10, may be produced using routine recombinant techniques or obtained from commercial suppliers (e.g. R&D Systems, Minneapolis, Minn.; Stemgent Inc, USA; Miltenyi Biotec Gmbh, DE).

SMAD1, SMAD5 and SMAD9 mediated intracellular signalling pathways may be stimulated by the second and third mesoderm induction media through the presence in the media of a second TGFβ ligand. The second TGFβ ligand may be a Bone Morphogenic Protein (BMP). Bone Morphogenic Proteins (BMPs) bind to Bone Morphogenic Protein Receptors (BMPRs) and stimulate intracellular signalling through pathways mediated by SMAD1, SMAD5 and SMAD9. Suitable Bone Morphogenic Proteins include any member of the BMP family, for example BMP2, BMP3, BMP4, BMP5, BMP6 or BMP7. Preferably the second TGFβ ligand is BMP2 (NCBI GeneID: 650, nucleic acid sequence NM_001200.2 GI: 80861484; amino acid sequence NP_001191.1 GI: 4557369) or BMP4 (NCBI GeneID: 652, nucleic acid sequence NM_001202.3 GI: 157276592; amino acid sequence NP_001193.2 GI: 157276593). Suitable BMPs include BMP4. Conveniently, the concentration of a Bone Morphogenic Protein, such as BMP2 or BMP4 in a medium described herein may be from 1 to 500 ng/ml, for example any of about 0.5, 1, 2, 3, 4, 5, 6, 7, 8, 9, 10, 11, 12, 13, 14, 15, 20, 25, 30, 35, 40, 45, 50, 100, 150, 200, 250, 300, 350, 400, 450, 500 ng/ml, preferably about 10 ng/ml. BMPs may be produced using routine recombinant techniques or obtained from commercial suppliers (e.g. R&D, Minneapolis, USA, Stemgent Inc, USA; Miltenyi Biotec Gmbh, DE).

The GSK3β inhibition activity of the third mesoderm induction medium may be provided by the presence of a GSK3β inhibitor in the medium. GSK3β inhibitors inhibit the activity of glycogen synthase kinase 3β (Gene ID 2932: EC2.7.11.26). Preferred inhibitors specifically inhibit the activity of glycogen synthase kinase 3β. Suitable inhibitors include CHIR99021 (6-((2-((4-(2,4-Dichlorophenyl)-5-(4-methyl-1H-imidazol-2-yl)pyrimidin-2-yl)amino)ethyl)amino)nicotinonitrile; Ring D. B. et al., Diabetes, 52:588-595 (2003)) alsterpaullone, kenpaullone, BIO(6-bromoindirubin-3′-oxime (Sato et al Nat Med. 2004 January; 10(1):55-63), SB216763 (3-(2,4-dichlorophenyl)-4-(1-methyl-1H-indol-3-yl)-1H-pyrrole-2,5-dione), Lithium and SB415286 (3-[(3-chloro-4-hydroxyphenyl)amino]-4-(2-nitrophenyl)-1H-pyrrole-2,5-dione; Coghlan et al Chem Biol. 2000 October; 7(10):793-803). In some preferred embodiments, the GSK3β inhibitor is CHIR99021.Suitable glycogen synthase kinase 3β inhibitors may be obtained from commercial suppliers (e.g. Stemgent Inc. MA USA; Cayman Chemical Co. MI USA; Selleckchem, MA USA). For example, the third mesoderm induction medium may contain 0.1 to 100 μM, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 95 μM, of a GSK3β inhibitor, such as CHIR99021, preferably about 10 μM.

In preferred embodiments, the first, second and third mesoderm induction media are chemically defined media. For example, the first mesoderm induction medium may consist of a chemically defined nutrient medium supplemented with an effective amount of activin, preferably activin A, for example 50 ng/ml activin A; the second mesoderm induction medium may consist of a chemically defined nutrient medium supplemented with an effective amount of activin preferably activin A, for example 5 ng/ml activin A, BMP, preferably BMP4, for example 10 ng/ml BMP4; and FGF, preferably bFGF (FGF2), for example 5 ng/ml bFGF; and the third mesoderm induction medium may consist of a chemically defined nutrient medium supplemented with an effective amount of activin preferably activin A, for example 5 ng/ml activin A, BMP, preferably BMP4, for example 10 ng/ml BMP4; FGF, preferably bFGF (FGF2), for example 5 ng/ml bFGF; and GSK3 inhibitor, preferably CHIR-99021 , for example 10 μM CHIR-99021.

A chemically defined medium (CDM) is a nutritive solution for culturing cells which contains only specified components, preferably components of known chemical structure. A CDM is devoid of undefined components or constituents which include undefined components, such as feeder cells, stromal cells, serum, and complex extracellular matrices, such as matrigel™ For example, a CDM does not contain stromal cells, such as OP9 cells, expressing Notch ligands, such as DLL1 or DLL4.

The chemically defined nutrient medium may comprise a chemically defined basal medium. Suitable chemically defined basal media include Iscove's Modified Dulbecco's Medium (IMDM), Ham's F12, Advanced Dulbecco's modified eagle medium (DMEM) (Price et al Focus (2003), 25 3-6), Williams E (Williams, G. M. et al Exp. Cell Research, 89, 139-142 (1974)), RPMI-1640 (Moore, G. E. and Woods L. K., (1976) Tissue Culture Association Manual. 3, 503-508) and StemPro™-34 PLUS (ThermoFisher Scientific).

The basal medium may be supplemented by serum-free culture medium supplements and/or additional components in the medium. Suitable supplements and additional components are described above and may include L-glutamine or substitutes, such as GlutaMAX-1™, ascorbic acid, monothiolglycerol (MTG), antibiotics such as penicillin and streptomycin, human serum albumin, for example recombinant human serum albumin, such as Cellastim™ (Merck/Sigma) and Recombumin™ (albumedix.com), insulin, transferrin and 2-mercaptoethanol. A basal medium may be supplemented with a serum substitute, such as Knockout Serum Replacement (KOSR; Invitrogen).

The iPSCs may be cultured in the first mesoderm induction medium for 1 to 12 hours, for example any of 1, 2, 3, 4, 5, 6, 7, 8, 9, 10, 11 or 12 hours, preferably about 4 hours; then cultured in the second mesoderm induction medium for 30 to 54 hours, for example any of 35, 36, 37, 38, 39, 40, 41, 42, 43, 44, 45, 46, 47, 48, 49 or 50 hours, preferably about 44 hours; and then cultured in the third mesoderm induction medium for 36 to 60 hours, for example any of 37, 38, 39, 40, 41, 42, 43, 44, 45, 46, 47, 48, 49, 50, 51, 52 or 53 hours, preferably about 48 hours to produce a population of mesodermal cells.

Mesoderm cells are partially differentiated progenitor cells that are committed to mesodermal lineages and are capable of differentiation under appropriate conditions into all cell types in the mesenchyme (fibroblast), muscle, bone, adipose, vascular and haematopoietic systems. Mesoderm cells may express one or more mesodermal markers. For example, the mesoderm cells may express any one, two, three, four, five, six or all seven of Brachyury, Goosecoid, Mixl1, KDR, FoxA2, GATA6 and PDGFαR.

In a second stage, mesoderm cells may be differentiated into haemogenic endothelial cells (HECs) by culturing the population of mesoderm cells under suitable conditions to promote haemogenic endothelial (HE) differentiation. For example, the iPSCs cells may be cultured in an HE induction medium.

A suitable HE induction medium may (i) stimulate cKIT receptor (CD117) and/or cKIT receptor (CD117) mediated signalling pathways and (ii) stimulate VEGFR and/or VEGFR mediated signalling pathways. For example, the HE induction medium may comprise SCF and VEGF.

Vascular endothelial growth factor (VEGF) is a protein factor of the PDGF family which binds to VEGFR tyrosine kinase receptors and stimulates vasculogenesis and angiogenesis. Suitable VEGFs include any member of the VEGF family, for example any one of VEGF-A to VEGF-D and PIGF. Preferably, the VEGF is VEGF-A (also known as VEGF, NCBI Gene ID: 7422, nucleic acid sequence NM_001025366.2, amino acid sequence NP_001020537.2). VEGF is readily available from commercial sources (e.g. R&D Systems, USA). Conveniently, the concentration of VEGF in an HE induction medium described herein may be from 1 to 100 ng/ml, for example any of about 2, 3, 4, 5, 6, 7, 8, 9, 10, 12, 15, 16, 17, 18, 19, 20, 25, 30, 35, 40, 45 or 50, 55, 60, 65, 70, 75, 80, 85, 90 or 95 ng/ml, preferably about 15 ng/ml.

In some examples of HE induction media, VEGF may be replaced by a VEGF activator or agonist that stimulates VEGFR mediated signalling pathways. Suitable VEGF activators are known in the art and include proteins, such as gremlin (Mitola et al (2010) Blood 116(18) 3677-3680) nucleic acids, such as shRNA (e.g. Turunen et al Circ Res. 2009 Sep, 11; 105(6):604-9), CRISPR-based plasmids (e.g. VEGF CRISPR activation plasmid; Santa Cruz Biotech, USA), antibodies and small molecules.

Stem cell factor (SCF) is a cytokine that binds to the KIT receptor (KIT proto-oncogene, receptor tyrosine kinase) (CD117; SCFR) and is involved in haematopoiesis. SCF (also called KITLG, NCBI GeneID: 4254) may have the reference nucleic acid sequence NM_000899.5 or NM_03994.5 and the reference amino acid sequence NP_000890.1 or NP_003985.5. SCF is readily available from commercial sources (e.g. R&D Systems, USA). Conveniently, the concentration of SCF in an HE induction medium described herein may be from 1 to 1000 ng/ml, for example any of about 10, 20, 30, 40, 50, 60, 70, 80, 90, 100, 110, 120, 130, 140, 150, 200, 250, 300, 350, 400, 450, 500, 600, 700, 800, 900 ng/ml, preferably about 100 ng/ml.

In preferred embodiments, the HE induction medium is a chemically defined medium. For example, the HE induction medium may consist of a chemically defined nutrient medium supplemented with effective amounts of VEGF, for example 15 ng/ml VEGF; and SCF, for example 100 ng/ml SCF.

Suitable chemically defined nutrient media are described above and include StemPro™-34 (ThermoFisher Scientific).

The mesoderm cells may be cultured in the HE induction medium for 2 to 6 days or 3 to 5 days, preferably about 4 days, to produce a population of HE cells.

Haemogenic endothelial cells (HECs) are partially differentiated endothelial progenitor cells that have hematopoietic potential and are capable of differentiation under appropriate conditions into haematopoietic lineages. HE cells may express CD34. In some embodiments, HECs may not express CD73 or CXCR4 (CD184). For example, the HE cells may have the phenotype CD34+CD73− or CD3430 CD73−CXCR4−.

In a third stage, haemogenic endothelial (HE) cells may be differentiated into haematopoietic progenitor cells (HPCs) by culturing the population of HE cells under suitable conditions to promote haematopoietic differentiation. For example, the HE cells may be cultured in a haematopoietic induction medium.

A suitable haematopoietic induction medium may stimulate the following (i) cKIT receptor (CD117) and/or cKIT receptor (CD117) mediated signalling pathways, (ii) VEGFR and/or VEGFR mediated signalling pathways, (iii) MPL (CD110) and/or MPL (CD110) mediated signalling pathways (iv) FLT3 and/or FLT3 mediated signalling pathways (v) IGF1R and/or IGF1R mediated signalling pathways (vi) SMAD1, 5 and 9 and/or SMAD1, 5 and 9 mediated signalling pathways (vii) Hedgehog and/or Hedgehog signalling pathways (viii) EpoR and/or EpoR mediated signalling pathway and (ix) AGTR2 and/or AGTR2 mediated signalling pathways. A suitable haematopoietic induction medium may also inhibit the AGTR1 (angiotensin II type 1 receptor (AT₁)) and/or AGTR1 (angiotensin II type 1 receptor (AT₁)) mediated signaling pathway. A suitable haematopoietic induction medium may also have interleukin (IL) activity and FGF activity.

For example, a haematopoietic induction medium may comprise the differentiation factors: VEGF, SCF, Thrombopoietin (TPO), Flt3 ligand (Flt3L), IL-3, IL-6, IL-7, IL-11, IGF-1, BMP, FGF, Sonic hedgehog (SHH), erythropoietin (EPO), angiotensin II, and an angiotensin II type 1 receptor (AT₁) antagonist. An example of a suitable haematopoietic induction medium is the Stage 3 medium shown in Table 1 below.

Thrombopoietin (TPO) is a glycoprotein hormone that regulates platelet production. TPO (also called THPO, NCBI Gene ID: 7066) may have the reference nucleic acid sequence NM_000460.4 and the reference amino acid sequence NP_000451.1. TPO is readily available from commercial sources (e.g. R&D Systems, USA; Miltenyi Biotec Gmbh, DE). Conveniently, the concentration of TPO in a haematopoietic induction medium described herein may be from 3 to 300 ng/ml, for example any of about 3, 5, 7, 9, 10, 13, 15, 17, 19, 20, 22, 25, 27, 30, 32, 35, 40, 50, 60, 70, 80, 90, 100, 110, 120, 130, 140, 150, 200, 250, 300, 350, 400, 450, 500, 600, 700, 800, 900 ng/ml preferably about 30 ng/ml.

Flt3 ligand (Fms-related tyrosine kinase 3 ligand or FLT3L) is a cytokine with haematopoietic activity which binds to the FLT3 receptor and stimulates the proliferation and differentiation of progenitor cells. Flt3 ligand (also called FLT3LG, NCBI GeneID: 2323) may have the reference nucleic acid sequence NM_001204502.2 and the reference amino acid sequence NP_001191431.1. Flt3 is readily available from commercial sources (e.g. R&D Systems, USA; Miltenyi Biotec Gmbh, DE). Conveniently, the concentration of Flt3 ligand in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90, 100, 125, 150, 175, 200, 225 or 250 ng/ml, preferably about 25 ng/ml.

Interleukins (ILs) are cytokines that play major roles in immune development and function. ILs in a haematopoietic induction medium may include IL-3, IL-6, IL-7, and IL-11.

IL-3 (also called IL3 or MCGF, NCBI GeneID: 3562) may have the reference nucleic acid sequence NM_000588.4 and the reference amino acid sequence NP_000579.2. IL-3 is readily available from commercial sources (e.g. R&D Systems, USA; Miltenyi Biotec Gmbh, DE). Conveniently, the concentration of IL-3 in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90, 100, 125, 150, 175, 200, 225 or 250 ng/ml, preferably about 25 ng/ml.

IL-6 (also called IL6 or HGF, NCBI GeneID: 3569) may have the reference nucleic acid sequence NM_000600.5 and the reference amino acid sequence NP_000591.5. IL-6 is readily available from commercial sources (e.g. R&D Systems, USA; Miltenyi Biotec Gmbh, DE). Conveniently, the concentration of IL-6 in a haematopoietic induction medium described herein may be from 0.1 to 100 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 95 ng/ml preferably about 10 ng/ml.

IL-7 (also called IL7, NCBI GeneID: 3574) may have the reference nucleic acid sequence NM_000880.4 and the reference amino acid sequence NP_000871.1. IL-7 is readily available from commercial sources (e.g. R&D Systems, USA; Miltenyi Biotec Gmbh, DE). Conveniently, the concentration of IL-7 in a haematopoietic induction medium described herein may be from 0.1 to 100 ng/ml, for example any of about 0.1, 0.25, 0.5, 1, 2, 3, 4, 5, 6, 7, 8, 9, 10, 11, 12, 13, 14, 15, 20, 25, 30, 35, 40, 45, 50, 60, 70, 80, 90 or 95 ng/ml, preferably about 10 ng/ml.

IL-11 (also called AGIF, NCBI GeneID: 3589) may have the reference nucleic acid sequence NM_000641.4 and the reference amino acid sequence NP_000632.1. IL-11 is readily available from commercial sources (e.g. R&D Systems, USA; Miltenyi Biotec Gmbh, DE). Conveniently, the concentration of IL-11 ligand in a haematopoietic induction medium described herein may be from 0.5 to 100 ng/ml, for example any of about 0.5, 0.75, 1, 2, 3, 4, 5, 6, 7, 8, 9, 10, 11, 12, 13, 14, 15, 20, 25, 30, 35, 40, 45, 50, 60, 70, 80, 90 or 95 ng/ml, preferably about 5 ng/ml.

Insulin-like growth factor 1 (IGF-1) is a hormone that binds to the tyrosine kinases IGF-1 receptor (IGF1R) and insulin receptor and activates the multiple signalling pathways. IGF-1(also called IGF or MGF, NCBI GeneID: 3479) may have the reference nucleic acid sequence NM_000618.5 and the reference amino acid sequence NP_000609.1. IGF-1 is readily available from commercial sources (e.g. R&D Systems, USA). Conveniently, the concentration of IGF-1 in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 17, 20, 22, 25, 27, 30, 35, 40, 45, 50, 60, 70, 80, 90, 100, 125, 150, 175, 200, 225 or 250 ng/ml, preferably about 25 ng/ml.

Sonic hedgehog (SHH) is a ligand of the hedgehog signalling pathway that regulates vertebrate organogenesis. SHH (also called TPT or HHG1, NCBI GeneID: 6469) may have the reference nucleic acid sequence NM_000193.4 and the reference amino acid sequence NP_000184.1. SHH is readily available from commercial sources (e.g. R&D Systems, USA; Miltenyi Biotec Gmbh, DE). Conveniently, the concentration of SHH in a haematopoietic induction medium described herein may be from 0.25 to 250 ng/ml, for example any of about 0.1, 0.25, 0.5, 1, 2, 3, 4, 5, 6, 7, 8, 9, 10, 11, 12, 13, 14, 15, 17, 20, 22, 25, 27, 30, 35, 40, 45, 50, 60, 70, 80, 90, 100, 125, 150, 175, 200, 225 or 250 ng/ml, preferably about 25 ng/ml.

Erythropoietin (EPO) is a glycoprotein cytokine that binds to the erythropoietin receptor (EpoR) and stimulates erythropoiesis. EPO (also called DBAL, NCBI GeneID: 2056) may have the reference nucleic acid sequence NM_000799.4 and the reference amino acid sequence NP_000790.2. EPO is readily available from commercial sources (e.g. R&D Systems, USA; PreproTech, USA). Conveniently, the concentration of EPO in haematopoietic induction medium described herein may be from 0.02 to 20 U/ml, for example any of about 0.01, 0.025, 0.05, 0.075, 0.1, 0.5, 0.75, 1.0, 1.5, 2.0, 2.5, 3, 4, 5, 6, 7, 8, 9, 10 13, 15, 17, or 19 U/ml, preferably about 2 U/ml.

Angiotensin II is a heptapeptide hormone that is formed by the action of angiotensin converting enzyme (ACE) on angiotensin I. Angiotensin II stimulates vasoconstriction. Angiotensin I and II are formed by the cleavage of angiotensinogen (also called AGT, NCBI GeneID: 183), which may have the reference nucleic acid sequence NM_000029.4 and the reference amino acid sequence NP_000020.1. Angiotensin II is readily available from commercial sources (e.g. R&D Systems, USA; Tocris, USA). Conveniently, the concentration of angiotensin II in a haematopoietic induction medium described herein may be from 0.05 to 50 ng/ml, for example any of about 0.01, 0.025, 0.05, 0.075, 0.1, 0.5, 0.75, 1.0, 1.5, 2.0, 2.5, 3, 4, 5, 6, 7, 8, 9, 10, 15, 20, 30, 40 or 50 ng/ml preferably about 5 ng/ml.

Angiotensin II type 1 receptor (AT₁) antagonists (ARBs) are compounds that selectively block the activation of AT₁ receptor (AGTR1; Gene ID 185). Suitable AT₁ antagonists include losartan (2-Butyl-4-chloro-1-{[2′-(1H-tetrazol-5-yl)-4-biphenylyl]methyl}-1H-imidazol-5-yl)methanol), valsartan ((2S)-3-Methyl-2-(pentanoyl{[2′-(1H-tetrazol-5-yl)biphenyl-4-yl]methyl}amino)butanoic acid), and telmisartan (4′[(1,4′-Dimethyl-2′-propyl[2,6′-bi-1H-benzimidazol]-1′-yl)methyl][1,1′-biphenyl]-2-carboxylic acid. In some preferred embodiments, the AT₁ antagonist is losartan. Suitable AT₁ antagonists may be obtained from commercial suppliers (e.g. Tocris, USA; Cayman Chemical Co. MI USA). Conveniently, the concentration of angiotensin II type 1 receptor (AT₁) antagonist in a haematopoietic induction medium described herein may be from 1 to 1000 μM, for example any of about 10, 20, 30, 40, 50, 60, 70, 80, 90, 100, 110, 120, 130, 140, 150, 160, 170, 180, 190, 200, 250, 300, 350, 400, 450, 500, 600, 700, 800, 900 ng/ml, preferably about 100 μM.

In preferred embodiments, the haematopoietic induction medium is a chemically defined medium. For example, the haematopoietic induction medium may consist of a chemically defined nutrient medium supplemented with effective amounts of VEGF, for example 15 ng/ml; SCF, for example 100 ng/ml; thrombopoietin (TPO), for example 30 ng/ml; Flt3 ligand (FLT3L), for example 25 ng./ml; IL-3, for example 25 ng/ml; IL-6, for example 10 ng/ml; IL-7, for example 10 ng/ml; IL-11, for example 5 ng/ml; IGF-1, for example 25 ng/ml; BMP, for example BMP4 at 10 ng/ml; FGF, for example bFGF at 5 ng/ml; Sonic hedgehog (SHH), for example 25 ng/ml; erythropoietin (EPO), for example 2 u/ml; angiotensin II, for example 10 μg/ml, and an angiotensin II type 1 receptor (AT₁) antagonist, for example losartan, at 100 μM. A suitable haematopoietic induction medium be devoid of other differentiation factors. For example, a haematopoietic induction medium may consist of a chemically defined nutrient medium supplemented with one or more differentiation factors, wherein the one or more differentiation factors consist of VEGF, SCF, Thrombopoietin (TPO), Flt3 ligand (Flt3L), IL-3, IL-6, IL-7, IL-11, IGF-1, BMP, FGF, Sonic hedgehog (SHH), erythropoietin (EPO), angiotensin II, and an angiotensin II type 1 receptor (AT₁) antagonist (i.e. the medium does not contain any differentiation factors other than VEGF, SCF, Thrombopoietin (TPO), Flt3 ligand (Flt3L), IL-3, IL-6, IL-7, IL-11, IGF-1, BMP, FGF, Sonic hedgehog (SHH), erythropoietin (EPO), angiotensin II, and an angiotensin II type 1 receptor (AT₁) antagonist).

Suitable chemically defined nutrient media are described above and include StemPro™-34 PLUS (ThermoFisher Scientific) or a basal medium such as IMDM supplemented with albumin, insulin, selenium transferrin, and lipids as described below.

The HE cells may be cultured in the haematopoietic induction medium for 8-21 days, for example any of about 9, 10, 11, 12, 13, 14, 15, 16, 17, 18, 19 or 20 days, preferably about 16 days, to produce the population of HPCs.

HPCs (also called hematopoietic stem cells) are multipotent stem cells that are committed to a hematopoietic lineage and are capable of further hematopoietic differentiation into all blood cell types including myeloid and lymphoid lineages, including monocytes, B cells and T cells. HPCs may express CD34. HPCs may co-express CD133, CD45 and FLK1 (also known as KDR or VEGFR2) and may be negative for expression of CD38 and other lineage specific markers. For example, HPCs may display the phenotype CD34+CD133+CD45+FLK1+CD38−.

Following the generation of HPCs from HE cells, a population of HPCs expressing one or more cell surface markers, such as CD34, may be purified, for example by magnetic activated cell sorting (MACS), before being subjected to further differentiation. For example, a population of CD34+ HPCs may be purified. The CD34+ HPCs may be purified after 8 days, for example 8, 9 or 10 days, culture in the HE induction medium. The CD34+ HPCs may be purified after 16 days of differentiation, for example on day 16, 17 or 18 of the differentiation method.

In a fourth stage, haematopoietic progenitor cells (HPCs) may be differentiated into progenitor T cells by culturing the population of HPCs under suitable conditions to promote lymphoid differentiation. For example, the haematopoietic progenitor cells may be cultured in a lymphoid expansion medium.

A lymphoid expansion medium is a cell culture medium that promotes the lymphoid differentiation of HPCs into progenitor T cells.

A suitable lymphoid expansion medium may (i) stimulate cKIT receptor (CD117; KIT receptor tyrosine kinase) and/or cKIT receptor (CD117; KIT receptor tyrosine kinase) mediated signalling pathways, (ii) stimulate MPL (CD110) and/or MPL (CD110) mediated signalling pathways (iii) FLT3 and/or FLT3 mediated signalling pathways and (iv) have interleukin (IL) activity. For example, a lymphoid expansion medium may comprise the differentiation factors SCF, FLT3L, TPO and IL7.

In preferred embodiments, the lymphoid expansion medium is a chemically defined medium. For example, the lymphoid expansion medium may consist of a chemically defined nutrient medium supplemented with effective amounts of the above differentiation factors. Suitable lymphoid expansion media are well-known in the art and include Stemspan™ SFEM II (Cat #9605; StemCell Technologies Inc, CA). with Stemspan™ lymphoid expansion supplement (Cat #9915; StemCell Technologies Inc, CA).

The HPCs may be cultured on a surface during differentiation into progenitor T cells. For example, the HPCs may be cultured on a surface of a culture vessel, bead or other biomaterial or polymer.

Preferably, the surface may be coated with a factor that stimulates Notch signalling, for example a Notch ligand, such as Delta-like 1 (DLL1) or Delta-like 4 (DLL4). Suitable Notch ligands are well-known in the art and available from commercial suppliers.

The surface may also be coated with an extracellular matrix protein, such as fibronectin, vitronectin, laminin or collagen and/or one or more cell surface adhesion proteins, such as VCAM1. In some embodiments, the surface for HPC culture may have a coating that comprises a factor that stimulates Notch signalling, for example a Notch ligand, such as DLL4, without the extracellular matrix protein or cell surface adhesion protein.

In some embodiments, the surface for HPC culture may have a coating that comprises a factor that stimulates Notch signalling, for example a Notch ligand, such as DLL4, an extracellular matrix protein, such as vitronectin, and a cell surface adhesion protein, such as VCAM1. The surface may be coated with an extracellular matrix protein, factor that stimulates Notch signalling and cell surface adhesion protein by contacting the surface with a coating solution. For example, the coating solution may be incubated on the surface under suitable conditions to coat the surface. Conditions may, for example, include about 2 hours at room temperature. Coating solutions comprising an extracellular matrix protein and a factor that stimulates Notch signalling are available from commercial suppliers (StemSpan™ Lymphoid Differentiation Coating Material; Cat #9925; Stem Cell Technologies Inc, CA).

The HPCs may be cultured in the lymphoid expansion medium on the substrate or surface for a time sufficient for the HPCs to differentiate into progenitor T cells. For example, the HPCs may be cultured for 2-6 weeks, 2 to 5 weeks or 2-4 weeks, preferably 3 weeks.

Progenitor T cells are multi-potent lymphopoietic progenitor cells that are capable of giving rise to αβ T cells, γδ T cells, tissue resident T cells and NK T cells. Progenitor T cells may commit to the αβ T cell lineage after pre-TCR selection in the thymus. Progenitor T cells may be capable of in vivo thymus colonization and may be capable of committing to the T cell lineage after pre-TCR selection in the thymus. Progenitor T cells may also be capable of maturation into cytokine-producing CD3⁺ T-cells.

Progenitor T cells may express CD5 and CD7 i.e. the progenitor T cells may have a CD5+CD7+ phenotype. Progenitor T cells may also co-express CD44, CD25 and CD2. For example, progenitor T cells may have a CD5+, CD7+CD44+, CD25+CD2+ phenotype. In some embodiments, progenitor T cells may also co-express CD45. Progenitor T cells may lack expression of CD3, CD4 and CD8, for example on the cell surface.

In a fifth stage, progenitor T cells may be matured into T cells by culturing the population of progenitor T cells under suitable conditions to promote T cell maturation. For example, the progenitor T cells may be cultured in a T cell maturation medium.

A T cell maturation medium is a cell culture medium that promotes the maturation of progenitor T cells into mature T cells. A suitable T cell maturation medium may (i) stimulate cKIT receptor (CD117; KIT receptor tyrosine kinase) and/or cKIT receptor (CD117; KIT receptor tyrosine kinase) mediated signalling pathways (ii) FLT3 and/or FLT3 mediated signalling pathways and (iii) have interleukin (IL) activity. For example, a T cell maturation medium may comprise the differentiation factors SCF, FLT3L, and IL7.

In preferred embodiments, the T cell maturation medium is a chemically defined medium. For example, the T cell maturation medium may consist of a chemically defined nutrient medium supplemented with effective amounts of the above differentiation factors. Suitable T cell maturation media are well-known in the art and include Stemspan™ SFEM II (Cat #9605; StemCell Technologies Inc, CA) with Stemspan™ T cell maturation supplement (Cat #9930; StemCell Technologies Inc, CA) and other media suitable for expansion of PBMCs and CD3+ cells, such as ExCellerate Human T cell expansion medium (R& D Systems, USA). Other suitable T cell maturation media may include a basal medium such as IMDM, supplemented with ITS, albumin and lipids, as described elsewhere herein and further supplemented with effective amounts of the above differentiation factors.

The progenitor T cells may be cultured on a surface. For example, the progenitor T cells may be cultured on a surface of a culture vessel, bead or other biomaterial or polymer.

Preferably, the surface may be coated with a factor that stimulates Notch signalling, for example a Notch ligand, such as Delta-like 1 (DLL1) or Delta-like 4 (DLL4). Suitable Notch ligands are well-known in the art and available from commercial suppliers. The surface may also be coated with an extracellular matrix protein, such as fibronectin, vitronectin, laminin or collagen and/or one or more cell surface adhesion proteins, such as VCAM1. Suitable coatings are well-known in the art and described elsewhere herein.

The progenitor T cells may be cultured in the T cell maturation medium on the substrate or surface for a time sufficient for the progenitor T cells to mature into T cells. For example, the progenitor T cells may be cultured for 1-4 weeks, preferably 2 or 3 weeks.

In some embodiments, the T cells produced by maturation of progenitor T cells may be double positive CD4+CD8+ T cells.

Progenitor T cells may be matured into T cells by the methods described above. T cells (also called T lymphocytes) are white blood cells that play a central role in cell-mediated immunity. T cells can be distinguished from other lymphocytes by the presence of a T cell receptor (TCR) on the cell surface. There are several types of T cells, each type having a distinct function.

T helper cells (T_{H} cells) are known as CD4⁺ T cells because they express the CD4 surface glycoprotein. CD4⁺ T cells play an important role in the adaptive immune system and help the activity of other immune cells by releasing T cell cytokines and helping to suppress or regulate immune responses. They are essential for the activation and growth of CD4+CD8+ T cells.

Cytotoxic T cells (T_{C} cells, CTLs, killer T cells, CD4+CD8+ T cells) are known as CD8⁺ T cells because they express the CD8 surface glycoprotein. CD8⁺ T cells act to destroy virus-infected cells and tumour cells. Most CD8⁺ T cells express TCRs that can recognise a specific antigen displayed on the surface of infected or damaged cells by a class I MHC molecule. Specific binding of the TCR and CD8 glycoprotein to the antigen and MHC molecule leads to T cell-mediated destruction of the infected or damaged cells.

T cells produced as described herein may be double positive CD4+CD8+ T cells or single positive CD4+ or CD8+ T cells.

T cells produced as described herein may be mature CD3+ T cells. For example, the cells may have a TCR+CD3+CD8+CD4+ phenotype. In some embodiments, T cells may also express CD45 and CD28.

T cells produced as described herein may be γδ T cells, αβ T cells or NKT cells.

In some preferred embodiments, the T cells produced as described herein are αβ T cells.

Following maturation of progenitor T cells (stage 5), the population of T cells may be predominantly double positive CD4+CD8+ T cells.

In a sixth stage, the population of TCR T cells may be activated and/or expanded to produce or increase the proportion of single positive CD4+ T cells, or more preferably single positive CD8+ T cells. Suitable methods for activating and expanding T cells are well-known in the art. For example, T cells may be exposed to a T cell receptor (TCR) agonist under appropriate culture conditions. Suitable TCR agonists include ligands, such as peptides displayed on a class I or II MHC molecule (MHC-peptide complexes) on the surface of a bead or an antigen presenting cell, such as a dendritic cell, and soluble factors, such as anti-TCR antibodies for example antibody CD28 antibodies, and multimeric MHC-peptide complexes, such as MHC-peptide tetramers, pentamers or dextramers.

Activation refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

An anti-TCR antibody may specifically bind to a component of the TCR, such as εCD3, αCD3 or αCD28. Anti-TCR antibodies suitable for TCR stimulation are well-known in the art (e.g. OKT3) and available from commercial suppliers (e.g. eBioscience CO USA). In some embodiments, T cells may be activated by exposure to anti-αCD3 antibodies and IL2, IL7 or IL15. More preferably, T cells are activated by exposure to anti-αCD3 antibodies and anti-αCD28 antibodies. The activation may occur in the presence or absence of CD14⁺ monocytes. The T cells may be activated with anti-CD3 and anti-CD28 antibody coated beads. For example, PBMCs or T cell subsets including CD4⁺ and/or CD8⁺ cells may be activated, without feeder cells (antigen presenting cells) or antigen, using antibody coated beads, for example magnetic beads coated with anti-CD3 and anti-CD28 antibodies, such as Dynabeads® Human T-Activator CD3/CD28 (ThermoFisher Scientific). In other embodiments, soluble tetrameric antibody complexes that bind CD3, CD28 and CD2 cell surface ligands, such as ImmunoCult™ Human CD3/CD28/CD2 T Cell Activator or Human CD3/CD28 T Cell Activator, may be used to activate the T cells. In other embodiments, T cells may be activated with an MHC-peptide complex, preferably a multimeric MHC-peptide complex, optionally in combination with an anti-CD28 antibody.

T cells expressing a chimeric antigen receptor may be activated using a soluble antigen to the receptor. The antigen may be in a multimeric form or on the surface of a bead and may optionally be used in conjunction with an anti-TCR antibody, such as an anti-CD28 antibody.

In some embodiments, double positive CD4+CD8+ T cells may be cultured in a T cell maturation medium as described herein supplemented with IL-15. The medium may be further supplemented with a T cell receptor (TCR) agonist, for example one or more anti-TCR antibodies, such as anti-αCD3 antibodies, and anti-αCD28 antibodies, as described above in order to activate and expand the population and produce single positive T cells.

The T cells may be cultured using any convenient technique to produce the expanded population. Suitable culture systems include stirred tank fermenters, airlift fermenters, roller bottles, culture bags or dishes, and other bioreactors, in particular hollow fibre bioreactors. The use of such systems is well-known in the art

T cells produced as described herein may express a heterologous antigen receptor, such as a T cell receptor (TCR) NK cell receptor or chimeric antigen receptor (CAR) that binds a target antigen. For example, the heterologous antigen receptor may bind specifically to cancer cells that express a tumor antigen or peptide fragment thereof. Because they are RAG inactivated (i.e. the RAG1 and/or RAG2 genes in the cells are knocked out by a deletion or other mutation), the T cells do not express endogenous TCR. The T cells may be useful for example in immunotherapy, as described below.

Following production, the population of T cells, for example (double positive) DP CD4+CD8+ cells, (single positive) SP CD4+ cells or SP CD8+ cells, may be isolated and/or purified. Any convenient technique may be used, including fluorescence-activated cell sorting (FACS) or magnetic-activated cell sorting using antibody coated magnetic particles (MACS).

The population of T cells, for example DP CD4+CD8+ cells, SP CD4+ cells or SP CD8+ cells, may be expanded and/or concentrated. Optionally, the population of T cells produced as described herein may be stored, for example by cryopreservation, before use.

A population of T cells may be admixed with other reagents, such as buffers, carriers, diluents, preservatives and/or pharmaceutically acceptable excipients. Suitable reagents are described in more detail below. A method described herein may comprise admixing the population of T cells with a pharmaceutically acceptable excipient.

Pharmaceutical compositions suitable for administration (e.g. by infusion), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable vehicles can be found in standard pharmaceutical texts, for example, Remington's Pharmaceutical Sciences, 18th edition, Mack Publishing Company, Easton, Pa., 1990.

In some preferred embodiments, the T cells, which may be DP CD4+CD8+ T cells, SP CD4+ T cells or preferably SP CD8+ T cells, may be formulated into a pharmaceutical composition suitable for intravenous infusion into an individual.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

An aspect of the invention provides a population of T cells, which may be for example DP CD4+CD8+ T cells, SP CD4+ T cells or SP CD8+ T cells, produced by a method described above. The T cells may be RAG inactivated and may lack functional endogenous TCR genes, so the T cells do not express endogenous TCRs. The T cells may express an exogenous TCR from heterologous encoding nucleic acid.

The population of T cells may be for use as a medicament. For example, a population of mature T cells as described herein may be used in cancer immunotherapy therapy, for example adoptive T cell therapy.

Adoptive cellular therapy or adoptive immunotherapy refers to the adoptive transfer of human T lymphocytes that express antigen receptors that are specific for target cells. For example, human T lymphocytes may express TCRs that are specific for antigens expressed on target cells and/or that are specific for peptide MHC complexes expressed on target cells or chimeric antigen receptors (CAR) or NKCR that are specific for antigens expressed on target cells.

This can be used to treat a range of diseases depending upon the target chosen, e.g., tumour specific antigens to treat cancer. Adoptive cellular therapy involves removing a portion of a donor's or the patient's cells, for example, white blood cells. The cells are then used to generate iPSCs in vitro and these iPSCs are used to efficiently generate T cells that are specific for antigens expressed on target cells and/or specific for peptide MHC complexes on target cells as described herein. The T cells may be expanded, washed, concentrated, and/or then frozen to allow time for testing, shipping and storage until a patient is ready to receive the infusion of cells.

Other aspects of the invention provide the use of a population of T cells as described herein, or produced according to the methods described herein, for the manufacture of a medicament for the treatment of cancer, a population of T cells as described herein, or produced according to the methods described herein, for the treatment of cancer, and a method of treatment of cancer comprising administering a population of T cells as described herein, or produced according to the methods described herein, to an individual in need thereof.

The population of T cells may be autologous i.e. the T cells are produced from iPSCs derived from cells originally obtained from the same individual to whom the T cells are subsequently administered (i.e. the donor and recipient individual are the same).

The population of T cells may be allogeneic i.e. the T cells may be produced from iPSCs derived from cells originally obtained from a different individual to the individual to whom the T cells are subsequently administered (i.e. the donor and recipient individual are different). Allogeneic refers to a graft derived from a different animal of the same species.

The donor and recipient individuals may be HLA matched to avoid GVHD and other undesirable immune effects, such as rejection. Alternatively, the donor and recipient individuals may not be HLA matched, or HLA genes in the cells from the donor individual may be modified, for example by gene editing, to remove any HLA mismatch with the recipient.

A suitable population of T cells for administration to a recipient individual may be produced by a method comprising providing an initial population of cells obtained from a donor individual, reprogramming the cells into iPSCs, inactivating RAG in the iPSCs and differentiating the RAG inactivated iPSCs into T cells that express an antigen receptor, such as an αβ TCR which binds specifically to cancer cells or antigen or peptide of antigen on cancer cells optionally presented in complex with MHC, in the recipient individual, as described herein.

Following administration of the T cells, the recipient individual may exhibit a T cell mediated immune response against cancer cells in the recipient individual. This may have a beneficial effect on the cancer condition in the individual.

As used herein, the terms “cancer,” “neoplasm,” and “tumour” are used interchangeably and, in either the singular or plural form, refer to cells that have undergone a malignant transformation that makes them pathological to the host organism.

Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumour, a “clinically detectable” tumour is one that is detectable on the basis of tumour mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient.

Cancer conditions may be characterised by the abnormal proliferation of malignant cancer cells and may include leukaemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, oesophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

Cancer cells within an individual may be immunologically distinct from normal somatic cells in the individual (i.e. the cancerous tumour may be immunogenic). For example, the cancer cells may be capable of eliciting a systemic immune response in the individual against one or more antigens expressed by the cancer cells. The tumour antigens that elicit the immune response may be specific to cancer cells or may be shared by one or more normal cells in the individual.

The cancer cells of an individual suitable for treatment as described herein may express the antigen and/or may be of correct HLA type to bind the heterologous TCR expressed by the T cells.

An individual suitable for treatment as described above may be a mammal. In preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

In some embodiments, the individual may have minimal residual disease (MRD) after an initial cancer treatment.

An individual with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci AS et al., eds., McGraw-Hill, New York, 2001. In some instances, a diagnosis of a cancer in an individual may include identification of a particular cell type (e.g. a cancer cell) in a sample of a body fluid or tissue obtained from the individual.

An anti-tumour effect is a biological effect which can be manifested by a reduction in the rate of tumour growth, decrease in tumour volume, a decrease in the number of tumour cells, a decrease in the number of metastases, an increase in life expectancy, or amelioration of various physiological symptoms associated with the cancerous condition. An “anti-tumour effect” can also be manifested by the ability of the peptides, polynucleotides, cells and antibodies, also T cells which may be obtained according to the methods of the present invention, as described herein in prevention of the occurrence of tumour in the first place

Treatment may be any treatment or therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment may also be prophylactic (i.e. prophylaxis). For example, an individual susceptible to or at risk of the occurrence or re-occurrence of cancer may be treated as described herein. Such treatment may prevent or delay the occurrence or re-occurrence of cancer in the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumour volume or morphology (for example, as determined using computed tomographic (CT), sonography, or other imaging method), a delayed tumour growth, a destruction of tumour vasculature, improved performance in delayed hypersensitivity skin test, an increase in the activity of T cells, and a decrease in levels of tumour-specific antigens. Administration of T cells modified as described herein may improve the capacity of the individual to resist cancer growth, in particular growth of a cancer already present in the subject and/or decrease the propensity for cancer growth in the individual.

The T cells or the pharmaceutical composition comprising the T cells may be administered to a subject by any convenient route of administration, whether systemically/peripherally or at the site of desired action, including but not limited to; parenteral, for example, by infusion. Infusion involves the administration of the T cells in a suitable composition through a needle or catheter. Typically, T cells are infused intravenously or subcutaneously, although the T cells may be infused via other non-oral routes, such as intramuscular injections and epidural routes. Suitable infusion techniques are known in the art and commonly used in therapy (see, e.g., Rosenberg et al., New Eng. J. of Med., 319:1676, 1988).

Typically, the number of cells administered is from about 10⁵ to about 10¹⁰ per Kg body weight, for example any of about 1, 2, 3, 4, 5, 6, 7, 8, or 9, ×10⁵, ×10⁸, ×10⁷, ×10⁸, ×10⁹, or ×10¹⁰ cells per individual, typically 2×10⁸ to 2×10¹⁰ cells per individual, typically over the course of 30 minutes, with treatment repeated as necessary, for example at intervals of days to weeks. It will be appreciated that appropriate dosages of the TCR αβ+ T cells, and compositions comprising the TCR αβ+ T cells, can vary from patient to patient. Determining the optimal dosage will generally involve the balancing of the level of therapeutic benefit against any risk or deleterious side effects of the treatments of the present invention.

The selected dosage level will depend on a variety of factors including, but not limited to, the activity of the particular cells, cytokine release syndrome (CRS), the route of administration, the time of administration, the rate of loss or inactivation of the cells, the duration of the treatment, other drugs, compounds, and/or materials used in combination, and the age, sex, weight, condition, general health, and prior medical history of the patient. The amount of cells and the route of administration will ultimately be at the discretion of the physician, although generally the dosage will be to achieve local concentrations at the site of action which achieve the desired effect without causing substantial harmful or deleterious side-effects.

While the T cells may be administered alone, in some circumstances the T cells may be administered in combination with the target antigen, APCs displaying the target antigen, CD3/CD28 beads, IL-2, IL-7 and/or IL15 to promote expansion in vivo of the population of T cells. Administration in combination may be by separate, simultaneous or sequential administration of the combined components.

The population of T cells may be administered in combination with one or more other therapies, such as cytokines e.g. IL-2, CD4+CD8+ chemotherapy, radiation and immuno-oncology agents, including checkpoint inhibitors, such as anti-B7-H3, anti-B7-H4, anti-TIM3, anti-KIR, anti-LAG3, anti-PD-1, anti-PD-L1, and anti-CTLA4 antibodies. Administration in combination may be by separate, simultaneous or sequential administration of the combined components.

The one or more other therapies may be administered by any convenient means, preferably at a site which is separate from the site of administration of the T cells.

Administration of T cells can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. Preferably, the T cells are administered in a single transfusion of any of 500 million, 1 billion, 2, 3, 4, 5, 6, 7, 8, 9, 10, 11, 12, 13, 14, 15 billion T cells for example at least 1×10⁹ T cells.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

Unless defined otherwise, all technical and scientific terms used herein have the same meaning as commonly understood by one of ordinary skill in the art to which this invention belongs. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range. The figures and tables of the disclosure also describe ranges, and discrete values, which may constitute an element of any of the methods disclosed herein. Concentrations described herein are determined at ambient temperature and pressure. This may be, for example, the temperature and pressure at room temperature or in within a particular portion of a process stream. Preferably, concentrations are determined at a standard state of 21° C. and 1 bar of pressure. The term “about” means a value within two standard deviations of the mean for any particular measured value.

As used herein and in the claims, the singular forms “a,” “and,” and “the” include plural reference unless the context clearly dictates otherwise. Thus, for example, reference to “a peptide chain” is a reference to one or more peptide chains and includes equivalents thereof known to those skilled in the art.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### Experimental

### Methods

### hiPSC Culture

iPSCs were routinely cultured in mTeSR1 (SCT) on Matrigel (BD Corning) using tissue culture plasticware, in 5% CO₂, 5% O₂ at 37° C. hiPSCs were harvested manually using an EasyPassage tool (Invitrogen) and cells seeded at 1:6 or 1:12 ratios, in media with Y27632 (R&D Systems) at 10 uM for the first 48 h of culture. For differentiation, hiPSCs were passaged onto either matrigel or vitronectin at low density cultures using 1:48 or 1:98 split ratio. Seeding density was about 1 colony per field of view, when viewed under ×4 magnification on the microscope, at 24 h post seeding. hiPSCs were cultured in mTeSR2 or E8 flex (SCT), depending on the cell culture matrix used, for approx. 4-5 days until colonies were compacted and distinct cells were no longer visible.

### T Cell Differentiation from Pluripotent Stem Cells

hiPSC maintenance medium (mTeSR2 or E8 flex) was removed and the cells were washed twice with DMEM/F12.

2 mL of StemPro34 PLUS (StemPro34 from Invitrogen; StemPro34 basal media, with supplement added and Penicillin Streptomycin (1% v/v: Invitrogen) and Glutamine (2 mM: Invitrogen), Ascorbic Acid (50 μg/ml: Sigma Aldrich) and monothioglycerol (100 μM: Sigma Aldrich), further supplemented with 50 ng/mL of Activin was added and incubated for 4 hours. Volumes are dependent on culture flask size, typically at least 2 mls/9 cm², and 20 mls /150 cm².

After 4 hours, the medium was removed and the cells were washed twice with DMEM/F12 to remove residual high concentration Activin A. The medium was replaced with 2 mL of StemPro34 PLUS supplemented with 5 ng/mL of Activin A, 10 ng/ml of BMP4 and 5 ng/ml of bFGF and incubated for 44 hours (Stage 1 media). The medium was then replaced with fresh Stage 1 media and supplemented with 10 μM CHIR-99021 and further cultured for 48 hours.

On Day 4, the medium was removed and the cells were washed twice with DMEM/F12 to remove residual stage 1 cytokines. The medium was then replaced with StemPro34 PLUS supplemented with 100 ng/mL of SCF and 15 ng/ml of VEGF and incubated for 48 hours (Stage 2 media). The medium was then replenished with fresh Stage 2 media and the cells cultured for a further 48 hours.

The medium was then replaced by the Stage 3 medium shown in Table 1 and the cells cultured for between 16-18 days, with 1:1 (v/v) feeding every 48 h. Typically this involved harvest of media and collection of cells in suspension by centrifugation (at 300 g, 10 min), and returning suspension cells to culture with fresh media (i.e. 20 mls for a T150 flask).

On approx. d16-18 depending on hiPSC line used, (confirmed separately by flow cytometry prior to day of harvest) CD34+ cells were isolated from the resulting monolayers for onward culture. Here, hiPSC lines designated ChiPSC31 (Takara), NIH2 (WT: a sub clone of MR1.1 from Lonza)) and sub-clones of NIH2: c3F3 and c1A12 were routinely included in experiments. CD34+ cells were harvested by sequential incubation with Accutase (SCT: for 30 min at 37° C.) and then Collagenase II (Invitrogen: 2 mg/ml) for 30 min at 37° C. Cell suspensions were collected and washed (×2 centrifugation at 300 g for 12 min in DMEM/F12), prior to CD34+ cell isolation via Magnetic activated beads (MACS) isolation (Miltenyi: according to manufacturer's instructions).

Following MACS isolation of CD34+ cells, these were then cryopreserved at 2×10⁵ cells/vial in CS10 (SCT), firstly at −80° C. by slow rate freezing, then in liquid nitrogen for long term storage.

For continued lymphoid proliferation and differentiation, Stem Cell Technologies proprietary 2 stage (Lymphoid Proliferation/T cell Maturation) media was employed (according to manufacturer's instructions).

### Generation of ADP-A2M4 Lentivirus

On the day prior to transfection, HEK293T (ATCC) cells were seeded at 40,000 cells per single stacker. The following day, reagents were pre-warmed as required (serum- and antibiotic-free RPMI (Life Technologies) and 293T media to 37° C., and Turbofect (Insight Biotechnology) and plasmids (gag/pol, REV, VSV-G from GenScript) to room temperature), and media from the single stackers was removed and replaced with fresh RPMI. In fresh falcon tubes, the transfection mix was prepared in the following order: serum- and antibiotic-free RPMI medium; packaging mix; lentivector plasmid at the required concentration based on production scale. Lastly, Turbofect transfection reagent was added carefully to the surface of the media, and the falcon was mixed by gently inverting 5 times before being added to culture flasks. Flasks were incubated at 37° C. for 48 hours.

Following 48 h incubation, the HEK293T culture supernatant was removed, centrifuged at 1400 rpm for 6 minutes to pellet cell debris and the supernatant was filtered once through 0.45 uM pore size filters (PES). Virus concentration was performed by centrifugation at 10,000 G for 16 hours at 4° C., with no brakes. Following concentration of the virus, supernatant was carefully removed from the virus pellet, and the viral pellet was resuspended in the desired amount of SFEM-II media. SFEM-II alone is the optimal media for progression of the differentiation through Stages 4-6 and reduces outgrowth of cell contaminants. ADP-A2M4 virus was snap-frozen and stored at −80° C. freezer.

### Transduction of T-Cell Progenitors With ADP-A2M4 TCR

iPSCs (CGT3 line) modified to prevent expression of RAG (termed RAG−/− or null) were cultured as described to generate CD34+ cells.

CD34+ cells, following MACS isolation, were cultured for 2 weeks in Stem Cell Technologies Lymphoid Proliferation media for 2 weeks to generate T-cell progenitors which expressed CD7 and CD5 (but not CD3, CD4 or CD8) indicating an immature cell type before TCR rearrangement would normally take place in an unedited line. Note lentiviral transduction may be performed at CD34+ isolation stage or iPS undifferentiated cell stage (e.g. mesoderm, HE (HECs), HPC, progenitor cells) and each have been shown to be successful.

A 1/32 dilution of virus particles (determined by biological titre experiments described above) in Lymphoid Progenitor media was added to the cells cultured in a 24 well plate (surface area 1.9 cm²) in a final volume of 500 μl.

Poloxamer (F108) (Sigma) was also added at a concentration of 1 mg/ml to improve the transduction efficiency. After 48 hours, a total media replacement was carried out and progenitor cells expanded into further/larger wells as appropriate. 1 week after transduction the cells were phenotyped by flow cytometry (BD Fortessa) using antibodies in Table 2 to check for mature T-cell marker expression.

### Results

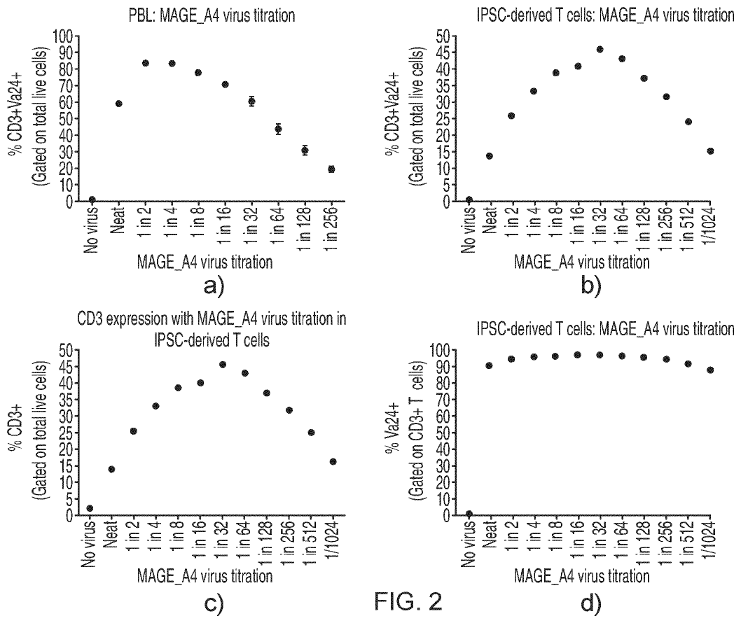

ADP-A2M4 lentivirus was titrated on both primary peripheral blood leukocytes (PBLs) and on stage 5 iPSC-derived T cells (NIH2). Data show the expected titration of TCR expression in PBL with increased dilution of ADP-A2M4 lentivirus (FIGS. 2A & 2B). Importantly, data suggests increased Vα24+ expression correlates with increased CD3+ expression with transduction of iPSC-derived T cells, indicating that LV transduction promotes surface expression of CD3 (FIGS. 2C & 1D).

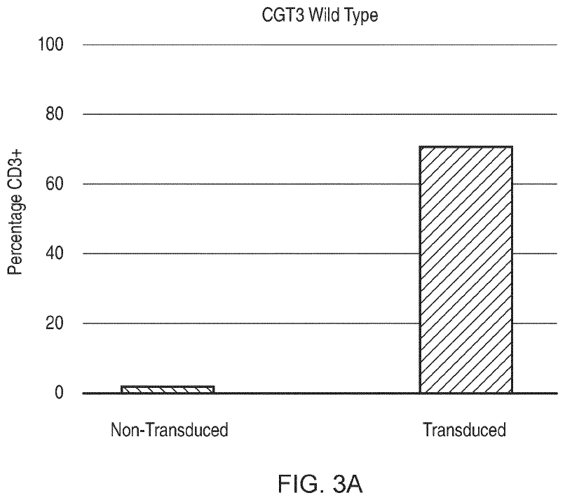

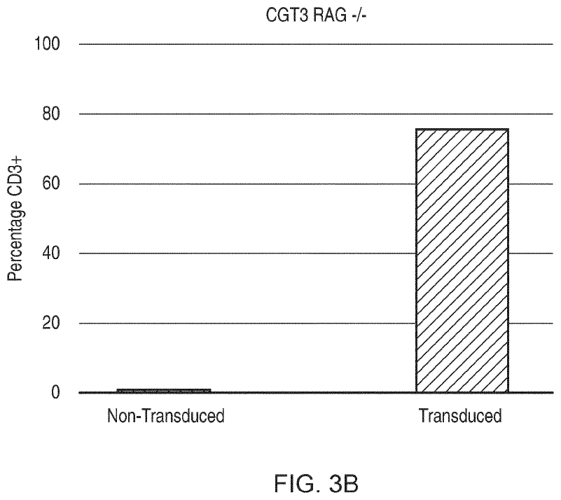

iPS cell line, CGT3, was differentiated using the T-cell differentiation protocol described herein and, after 2 weeks in lymphoid progenitor media, cells were transduced with ADP-A2M4 TCR. In non-transduced wild type line, a small number of cells successfully transition through selection stages and become CD3+, this number was found to be markedly improved with expression of the ADP-A2M4 TCR (FIG. 3A). In a RAG null genetic background, where no endogenous TCR gene rearrangement could occur, the CD3+ expression was negligible as complex formation with TCR is required for transport to the cell surface (FIG. 3B). This CD3 negative phenotype was shown to be rescued by lentiviral transduction of the ADP-A2M4 TCR (FIG. 3B).

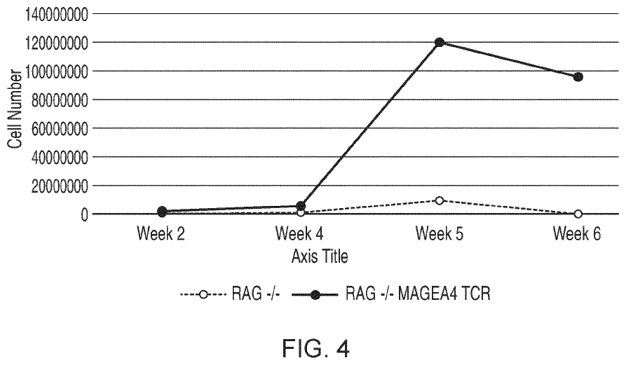

T-cell progenitor cells at the CD7/5+ stage were transduced with ADP-A2M4 TCR and cell counts performed at regular intervals. The cell numbers indicated greater proliferation and survival of T-cell progenitors after exogenous expression of a TCR to rescue the RAG null phenotype (FIG. 4).

TABLE 1

| Reagent | Final Concentration | |
|---|---|---|
| StemPro34 PLUS | 1 L (2 bottles required) | |
| VEGF | 15 | ng/mL |
| SCF | 100 | ng/mL |
| TPO | 30 | ng/mL |
| Flt3L | 25 | ng/mL |
| IL-3 | 25 | ng/mL |
| IL-6 | 10 | ng/mL |
| IL-7 | 10 | ng/mL |
| IL-11 | 5 | ng/mL |
| IGF-1 | 25 | ng/mL |
| BMP-4 | 10 | ng/mL |
| bFGF | 5 | ng/mL |
| SHH | 25 | ng/mL |
| EPO | 2 | U/mL |
| Angiotensin II | 10 | μg/mL |
| Losartan | 100 | μM |

TABLE 2
Antibodies used to phenotype T cells.

| Antibody | Volume/test (μl) |
|---|---|
| TCRaβ (IP26) PE (BioLegend: 306708) | 2.5 μl |
| TCRγδ (B1) APC (BioLegend: 331212) | 5 μl |
| CD5 (UCHT2) BV421 (BD: 562646) | 5 μl |
| CD7 (CD7-6B7) PerCP Cy5.5 (BioLegend: 343116) | 5 μl |
| CD45 (HI30) BUV395 (BD: 563792) | 5 μl |
| CD4 (OKT4) BV786 (BioLegend: 317442) | 5 μl |
| CD3 (SK7) AF488 (BioLegend: 344810) | 5 μl |
| CD8α (RPA-T8) PE-Cy7 (BD: 557746) | 5 μl |
| CD56 (NCAM16.2) BV605 (BD: 562780) | 5 μl |
| Ef506 BV510 (Invitrogen: 65-0866-14) | 1/100 dilution |

1. A method of producing a population of T cells comprising;
(i) differentiating a population of RAG inactivated induced pluripotent stem cells (iPSCs) into mesoderm cells
(ii) differentiating the mesoderm cells (MCs) to produce a population of haemogenic endothelial cells (HECs),
(iii) differentiating the HECs into a population of haematopoietic progenitor cells (HPCs),
(iv) differentiating the population of HPCs into progenitor T cells; and
(v) maturing the progenitor T cells to produce a population of double positive CD4+CD8+ T cells,
wherein the method comprises introducing a heterologous nucleic acid encoding an antigen receptor into one of the (a) iPSCs, (b) the HPCs or (c) the progenitor T cells.

2. A method according to claim 1 further comprising;
(vi) activating and expanding the T cells to produce a population of single positive CD8+ T cells or a population of single positive CD4+ T cells.

3. A method according to claim 1 or claim 2 wherein the method comprises introducing the heterologous nucleic acid into the iPSCs.

4. A method according to claim 1 or claim 2 wherein the method comprises introducing the heterologous nucleic acid into the HPCs.

5. A method according to claim 1 or claim 2 wherein the method comprises introducing the heterologous nucleic acid into the progenitor T cells.

6. A method according to any one of the preceding claims wherein the heterologous nucleic acid encoding the antigen receptor is comprised in an expression vector.

7. A method according to claim 6 wherein the expression vector is a lentiviral vector or adeno-associated viral (AAV) vector.

8. A method according to claim 6 wherein the lentiviral vector is a VSVg-pseudotyped viral vector.

9. A method according to any one of the preceding claims wherein the antigen receptor is a T cell receptor (TCR).

10. A method according to claim 9 wherein the TCR is an affinity enhanced TCR.

11. A method according to claim 9 or claim 10 wherein the TCR is a αβ TCR or γ67 TCR.

12. A method according to any one of claims 9 to 11 wherein the TCR binds specifically to an MHC displaying a peptide fragment of a target antigen expressed by cells or specifically binds to a target antigen or peptide thereof expressed by cells independently of MHC presentation.

13. A method according to claim 12 wherein the TCR binds specifically to an MHC displaying a peptide fragment of a tumour antigen expressed by the cancer cells or binds specifically to a tumour antigen or peptide fragment thereof expressed by cancer cells independently of MHC presentation.

14. A method according to any one of claims 1 to 8 wherein the antigen receptor is a chimeric antigen receptor (CAR).

15. A method according to claim 14 wherein the CAR binds specifically to a target antigen expressed by cells.

16. A method according to claim 15 wherein the TCR binds specifically to a tumour antigen expressed by the cancer cells.

17. A method according to any one of claims 1 to 8 wherein the antigen receptor is an NK cell receptor (NKCR).

18. A method according to claim 17 wherein the NKCR binds specifically to an MHC displaying a peptide fragment of a target antigen expressed by cells.

19. A method according to claim 18 wherein the NKCR receptor binds specifically to an MHC displaying a peptide fragment of a tumour antigen expressed by the cancer cells.

20. A method according to any one of claim 13, 16 or 19 wherein the tumour antigen is alpha-fetoprotein (AFP), NY-ESO1, MAGE-A10 or MAGE-A4.

21. A method according to any one of the preceding claims wherein the RAG inactivated iPSCs are differentiated into mesoderm cells by culturing the population of iPSCs under suitable conditions to promote mesodermal differentiation.

22. A method according to any one of the preceding claims wherein the RAG inactivated iPSCs are cultured sequentially in first, second and third mesoderm induction media to induce differentiation into mesoderm cells.

23. A method according to claim 22 wherein the first mesoderm induction medium stimulates SMAD2 and SMAD3 mediated signalling pathways.

24. A method according to claim 23 wherein the first mesoderm induction medium comprises activin.

25. A method according to claim 23 or claim 24 wherein the first mesoderm induction medium consists of a chemically defined nutrient medium supplemented with one or more differentiation factors, wherein the one or more differentiation factors consist of activin.

26. A method according to any one of claims 15 to 18 wherein the second mesoderm induction medium (i) stimulates SMAD1, SMAD2, SMAD3, SMAD5 and SMAD9 mediated signalling pathways and (ii) has fibroblast growth factor (FGF) activity.

27. A method according to claim 26 wherein the second mesoderm induction medium comprises activin, BMP, and FGF.

28. A method according to claim 26 or claim 27 wherein the second mesoderm induction medium consists of a chemically defined nutrient medium supplemented with one or more differentiation factors, wherein the one or more differentiation factors consist of activin, BMP, and FGF.

29. A method according to any one of claims 22 to 28 wherein the third mesoderm induction medium (i) stimulates SMAD1, SMAD2, SMAD3, SMAD5 and SMAD9 mediated signalling pathways (ii) has fibroblast growth factor (FGF) activity and (iii) inhibits glycogen synthase kinase 3β.

30. A method according to claim 29 wherein the third mesoderm induction medium comprises activin, BMP, FGF, and a GSK3 inhibitor.

31. A method according to claim 30 wherein the third mesoderm induction medium consists of a chemically defined nutrient medium supplemented with one or more differentiation factors, wherein the one or more differentiation factors consist of activin, BMP, FGF, and a GSK3 inhibitor.

32. A method according to any one of the preceding claims wherein the mesoderm cells display one or more of Brachyury+Goosecoid+Mixl1+KDR+FoxA2+GATA6+ and PDGFαR+.

33. A method according to any one of the preceding claims wherein the mesoderm cells are differentiated into HECs by culturing the population of mesoderm cells under suitable conditions to promote haemogenic endothelial (HE) differentiation.

34. A method according to any one of the preceding claims wherein the mesoderm cells are cultured in an HE induction medium to induce differentiation into HECs.

35. A method according to claim 34 wherein the HE induction medium (i) stimulates KIT (KIT proto-oncogene, receptor tyrosine kinase) mediated signalling pathways and (ii) stimulates VEGFR mediated signalling pathways.

36. A method according to claim 35 wherein the HE induction medium comprises SCF and VEGF.

37. A method according to claim 36 wherein the HE induction medium consists of a chemically defined nutrient medium supplemented with one or more differentiation factors, wherein the one or more differentiation factors consist of SCF and VEGF.

38. A method according to any one of the preceding claims wherein the HPCs are differentiated into progenitor T cells under suitable conditions to promote lymphoid differentiation.

39. A method according to claim 38 wherein the HPCs are differentiated by a method comprising culturing the population of HPCs in a lymphoid expansion medium to produce the progenitor T cells.

40. A method according to claim 38 or 39 wherein the progenitor T cells have a CD5+, CD7+ phenotype.

41. A method according to any one of the preceding claims wherein the progenitor T cells are matured into T cells under suitable conditions to promote T cell maturation.

42. A method according to claim 41 wherein the progenitor T cells are matured by a method comprising culturing the population of progenitor T cells in a T cell maturation medium to produce the double positive T cells.

43. A method according to any one of the preceding claims further comprising isolating or purifying the double positive T cells and/or single positive T cells.

44. A method according to claim 43 wherein double positive T cells and/or single positive T cells are isolated by magnetic activated cell sorting.

45. A method according to according to any one of the preceding claims comprising concentrating the population of double positive T cells and/or single positive T cells.

46. A method according to according to any one of the preceding claims comprising storing the population of double positive T cells and/or single positive T cells.

47. A method according to any one of the preceding claims comprising formulating the population of double positive T cells and/or single positive T cells with a pharmaceutically acceptable excipient.

48. A population of double positive T cells and/or single positive T cells produced by a method according to any one of claims 1 to 47.

49. A population according to claim 48 wherein the T cells express a heterologous antigen receptor.

50. A population according to claim 49 wherein the heterologous antigen receptor is a TCR, CAR or NKCR.

51. A population according to claim 49 or claim 50 wherein the T cells express are RAG inactivated and do not express an endogenous TCR.

52. A pharmaceutical composition comprising a population of double positive T cells and/or single positive T cells produced by a method according to any one of claims 1 to 47 and a pharmaceutically acceptable excipient.

53. A population of double positive T cells and/or single positive T cells produced by a method according to any one of claims 1 to 47 for use in a method of treatment.

54. A population of double positive T cells and/or single positive T cells produced by a method according to any one of claims 1 to 47 for use in a method of treatment of cancer.

55. A method of treatment of cancer comprising administering a population of double positive T cells and/or single positive T cells produced by a method according to any one of claims 1 to 47 to an individual in need thereof.
